(19) United States
(12) Patent Application Publication
(10) Pub. No.: US 2005/0148593 A1
(43) Pub. Date: Jul. 7, 2005

(54) Compounds, compositions, and methods

(76) Inventor: Gustave Bergnes, Pacifica, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/982,195

(22) Filed: Nov. 5, 2004

Related U.S. Application Data
(60) Provisional application No. 60/518,033, filed on Nov. 7, 2003.

(51) Int. Cl. A61K 31/525; A61K 31/519; C07D 487/02; C07D 475/04

(52) U.S. Cl. 514/251; 514/264.11; 544/279; 544/259

(57) ABSTRACT

Compounds, compositions and methods useful for treating cellular proliferative diseases and disorders by modulating the activity of KSP are disclosed.

This application claims the benefit of priority to U.S. Provisional Patent Application Ser. No. 60/518,033, filed Nov. 7, 2003, which is hereby incorporated by reference.

This invention relates to quinazolinone-like derivatives that are inhibitors of the mitotic kinesin KSP and are useful in the treatment of cellular proliferative diseases, for example cancer, hyperplasias, restenosis, cardiac hypertrophy, immune disorders and inflammation.

The mitotic spindle is responsible for distribution of replicate copies of the genome to each of the two daughter cells that result from cell division. Disruption of the mitotic spindle can inhibit cell division, and induce cell death. Microtubules are the primary structural element of the mitotic spindle; they are the site of action of certain existing therapeutic agents used to treat cancer, such as taxanes and vinca alkaloids. Microtubules, however, exist as elements in other types of cellular structures (including tracks for intracellular transport in nerve processes). The therapeutic targeting of microtubules can, therefore, modulate processes in addition to cellular proliferation, leading to side effects that limit the usefulness of such drugs.

Improvement in the specificity of agents used to treat cancer is of considerable interest because of the therapeutic benefits that would be realized if the side effects associated with the administration of these agents could be reduced. Dramatic improvements in the treatment of cancer have been associated with identification of therapeutic agents acting through novel mechanisms. Examples of this include not only the taxanes, but also the camptothecin class of topoisomerase I inhibitors.

One novel anti-proliferative mechanism entails selective inhibition of mitotic kinesins, enzymes that are essential for assembly and function of the mitotic spindle, but are not generally part of other microtubule structures, such as in nerve processes. See, e.g., Guidebook to the Cytoskeletal and Motor Proteins, Kreis and Vale, Eds., pp. 389-394 (Oxford University Press 1999). Mitotic kinesins play essential roles during all phases of mitosis. These enzymes are “molecular motors” that transform energy released by hydrolysis of ATP into mechanical force that drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death. Mitotic kinesins are attractive targets for the discovery and development of novel anti-mitotic chemotherapeutics.

Among the mitotic kinesins that have been identified is KSP. KSP belongs to an evolutionarily conserved kinesin subfamily of plus end-directed microtubule motors that assemble into bipolar homotetramers consisting of antiparallel homodimers. During mitosis, KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focusing of microtubules at the spindle pole.

Human KSP (also termed HsEg5) has been described [Blangy, et al., Cell, 83: 1159-69 (1995); Whitehead, et al., Arthritis Rheum., 39: 1635-42 (1996); Galgio et al., J. Cell Biol., 135: 339-414 (1996); Blangy, et al., J. Biol. Chem., 272: 19418-24 (1997); Blangy, et al., Cell Motil. Cytoskeleton, 40: 174-82 (1998); Whitehead and Rattner, J. Cell Sci., 111: 2551-61 (1998); Kaiser, et al., JBC 274: 18925-31 (1999); GenBank accession numbers: X85137, NM004523 and U37426], and a fragment of the KSP gene (TRIP5) has been described [Lee, et al., Mol. Endocrinol., 9: 243-54 (1995); GenBank accession number L40372]. Xenopus KSP homologs (Eg5), as well as Drosophila KLP61 F/KRP1 30 have been reported.

The present invention provides compounds, compositions and methods useful in the inhibition of mitotic kinesins, such as KSP (such as human KSP). The compounds can be used to treat cellular proliferative diseases and include certain heterocyclic-fused pyrimidinone derivatives. The invention also provides pharmaceutical formulations comprising one or more compounds of Formula I, and methods of treatment employing such compounds or compositions.

In one aspect, the invention provides at least one chemical entity chosen from a compound of Formula I: 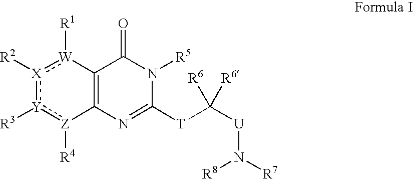
and pharmaceutically acceptable salts, solvates, crystal forms, diastereomers, and prodrugs thereof, wherein:
T and U are independently absent or optionally substituted lower alkylene;
W, X and Y are independently chosen from —N═, —N—, —C═, CH, CRⁱ, O and S;
Z is chosen from —N═, —N—, —C═, CH, CRⁱ, O and S, or is absent, provided that:
   no more than two of W, X, Y and Z are both —N═, O or S, and
   W, X, Y or Z can be O or S only when the ring they form is not aromatic, or W, X or Y can be O or S when Z is absent;
Rⁱ is chosen from optionally substituted alkyl, optionally substituted alkoxy, optionally substituted aryl, optionally substituted heteroaryl, halo and cyano;
R¹, R², R³ and R⁴ are independently chosen from hydrogen, hydroxy, optionally substituted alkyl (including, but not limited to, fluoroalkyl, sulfonamidoalkyl and carboxyalkyl), optionally substituted alkoxy, alkylsulfonyl, alkylsulfonamido, carboxamido, alkylthio, aminocarbonyl, optionally substituted aryl, optionally substituted heteroaryl, halo, nitro and cyano, provided that R¹, R² or R³ is absent when W, X or Y, respectively, is —N═, S, or O, and R⁴ is absent when Z is —N═, S, or O or is absent;
R⁵ is chosen from optionally substituted alkyl, optionally substituted aryl and optionally substituted heteroaryl;
R⁶ is chosen from optionally substituted alkyl and optionally substituted aryl,
R^{6′} is chosen from hydrogen, optionally substituted alkyl and optionally substituted aryl;
R⁷ is chosen from optionally substituted alkyl, optionally substituted aryl and optionally substituted heteroaryl; and
R⁸ is chosen from hydrogen, —C(O)—R⁹, —S(O)₂—R^{9′}, —CH₂—R⁹, —C(O)—O—R^{9′}, —C(O)—NH—R⁹ or —S(O)₂—NH—R⁹, wherein:
   R⁹ is chosen from hydrogen, optionally substituted alkyl, optionally substituted aryl and optionally substituted heteroaryl, and
   R^{9′} is chosen from optionally substituted alkyl, optionally substituted aryl and optionally substituted heteroaryl;
or alternatively:
R⁶ and R^{6′}, taken together with the carbon to which they are bonded, form an optionally substituted cycloalkyl or optionally substituted heterocycloalkyl having 5 to 7 ring atoms; or
R⁶ and R⁷ taken together with the nitrogen to which R⁷ is bonded form an optionally substituted 5- to 12-membered heterocycle having up to 2 additional heteroatoms selected from O, N and S, provided that such heterocycle is not optionally substituted: 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl; or
R⁷ and R⁸, taken together with the nitrogen to which they are bonded form an optionally substituted 5- to 12-membered heterocycle having up to 2 additional heteroatoms selected from O, N and S, provided that such heterocycle is not optionally substituted: 1-piperazin-1-yl, 1-[1,4]diazepan-1-yl, 2-oxo-[1,4]diazepan-1-yl, 7-oxo-[1,4]diazepan-1-yl, imidazol-1-yl or imidazolin-1-yl; or
R⁶ taken together with R⁷, and R^{6′} taken together with R⁸ and the nitrogen to which R⁷ and R⁸ are bonded form an optionally substituted 10- to 13-membered fused heterocycle having up to 2 additional heteroatoms selected from O, N and S,
   provided that:
at least one of W, X, Y or Z is other than —C═; and
when T is a covalent bond, then Z is present and at least one of W, X, Y or Z is O or S; and
when T is a covalent bond and R⁸ is hydrogen, —C(O)—R⁹, —S(O)₂—R^{9′}, —CH₂—R⁹, —C(O)—O—R^{9′}, —C(O)—NH—R⁹ or —S(O)₂—NH—R⁹, and when R⁷ and R⁸ are optionally substituted imidazolyl or optionally substituted imidazolinyl, then at least one of W, X, Y or Z is —N—, CH, CRⁱ, O or S.
   The dashed lines in Formula I indicate that the corresponding bond may be a single bond (e.g., where X is CH) or a double bond (e.g., where X is —C═). Chemical entities described herein are useful as active agents in the practice of the methods of treatment and in manufacture of compositions including the pharmaceutical formulations provided herein, and may also be useful as intermediates in the synthesis of such active agents.

In one of its aspects, the present invention provides compounds, methods and compositions employing at least one chemical entity chosen from a compound of Formula I, and pharmaceutically acceptable salts, solvates, crystal forms, diastereomers, and prodrugs thereof, wherein
T is optionally substituted lower alkylene;
U is chosen from a covalent bond or optionally substituted lower alkylene;
W, X and Y are independently chosen from —N═, N, —C═, CH, CRⁱ, O and S;
Z is chosen from —N═, N, —C═, CH, CRⁱ, O and S, or is absent, provided that:
   no more than two of W, X, Y and Z are both —N═, O or S, and
   W, X, Y or Z can be O or S only when the ring they form is not aromatic, or W, X or Y can be O or S when Z is absent;
Rⁱ is chosen from optionally substituted alkyl, optionally substituted alkoxy, optionally substituted aryl, optionally substituted heteroaryl, halogen and cyano;
R¹, R², R³ and R⁴ are independently chosen from hydrogen, hydroxy, optionally substituted alkyl (including, but not limited to, fluoroalkyl, sulfonamidoalkyl and carboxyalkyl), optionally substituted alkoxy, alkylsulfonyl, alkylsulfonamido, carboxamido, alkylthio, aminocarbonyl, optionally substituted aryl (including, but not limited to, sulfonamidoaryl), optionally substituted heteroaryl, halogen, nitro and cyano, provided that R¹, R², or R³ is absent where W, X or Y, respectively, is —N═S, or O, and R⁴ is absent where Z is —N═, S, O or is absent;
R⁵ is chosen from optionally substituted alkyl, optionally substituted aryl and optionally substituted heteroaryl;
R⁶ is chosen from optionally substituted alkyl and optionally substituted aryl,
R^{6′} is chosen from hydrogen, optionally substituted alkyl and optionally substituted aryl;
R⁷ is chosen from optionally substituted alkyl, optionally substituted aryl and optionally substituted heteroaryl; and
R⁸ is chosen from hydrogen, —C(O)—R⁹, —S(O)₂—R^{9′}, —CH₂—R⁹, —C(O)—O—R^{9′}, —C(O)—NH—R⁹ or —S(O)₂—NH—R⁹, in which:
   R⁹ is chosen from hydrogen, optionally substituted alkyl, optionally substituted aryl and optionally substituted heteroaryl, and
   R^{9′} is chosen from optionally substituted alkyl, optionally substituted aryl and optionally substituted heteroaryl;
or alternatively:
R⁶ taken together with R^{6′} is optionally substituted cycloalkyl or optionally substituted heterocycloalkyl having 5 to 7 ring atoms;
R⁶ and R⁷ taken together with the nitrogen to which R⁷ is bonded form an optionally substituted 5 to 12 membered heterocycle having up to 2 additional heteroatoms selected from O, N and S; or
R⁷ and R⁸, taken together with the nitrogen to which they are bonded form an optionally substituted 5 to 12 membered heterocycle having up to 2 additional heteroatoms selected from O, N and S; or
R⁶ taken together with R⁷, and R^{6′} taken together with R⁸ and the nitrogen to which R⁷ and R⁸ are bonded form an optionally substituted 10 to 13-membered fused heterocycle having up to 2 additional heteroatoms selected from O, N and S.

In one aspect, the invention provides methods of treating cellular proliferative diseases, treating disorders treatable by modulating KSP kinesin activity, and inhibiting KSP kinesin by the administration of a therapeutically effective amount of at least one chemical entity chosen from a compound of Formula I and pharmaceutically acceptable salts, solvates, crystal forms, diastereomers, and prodrugs thereof. Diseases and disorders that respond to therapy with such chemical entities include cancer, hyperplasia, restenosis, cardiac hypertrophy, immune disorders, inflammation, and the like.

In another aspect, the invention provides pharmaceutical compositions comprising a therapeutically effective amount of at least one chemical entity chosen from a compound of Formula I and pharmaceutically acceptable salts, solvates, crystal forms, diastereomers, and prodrugs thereof, and at least one pharmaceutically acceptable excipient.

In yet another aspect, the invention provides kits comprising at least one chemical entity chosen from a compound of Formula I and pharmaceutically acceptable salts, solvates, crystal forms, diastereomers, and prodrugs thereof I and a package insert or other labeling including directions for treating a cellular proliferative disease by administering a KSP kinesin inhibitory amount of at least one chemical entity chosen from a compound of Formula I and pharmaceutically acceptable salts, solvates, crystal forms, diastereomers, and prodrugs thereof.

In an additional aspect, the present invention provides methods of screening for compounds that will bind to a KSP kinesin, for example compounds that will displace or compete with the binding of the compounds of the invention. The methods comprise combining a labeled compound of the invention, a KSP kinesin, and at least one candidate agent and determining the binding of the candidate agent to the KSP kinesin.

In a further aspect, the invention provides methods of screening for modulators of KSP kinesin activity. The methods comprise combining a compound of the invention, a KSP kinesin, and at least one candidate agent and determining the effect of the candidate agent on the KSP kinesin activity.

The present invention provides compounds, compositions and methods useful in the inhibition of mitotic kinesins, such as KSP (for example, human KSP). The compounds can be used to treat cellular proliferative diseases and include certain heterocyclic-fused pyrimidinone derivatives.

As used in the present specification, the following words and phrases are generally intended to have the meanings as set forth below, except to the extent that the context in which they are used indicates otherwise. The following abbreviations and terms have the indicated meanings throughout:
Ac=acetyl
Boc=t-butyloxy carbonyl
Bu=butyl
c-=cyclo
CBZ=carbobenzoxy=benzyloxycarbonyl
DCM=dichloromethane=methylene chloride=CH₂Cl₂
DCE=dichloroethane
DIEA=N,N-diisopropylethylamine
DMF=N,N-dimethylformamide
DMSO=dimethyl sulfoxide
Et=ethyl
EDC=ethyldiethylaminopropylcarbodiimide
Fmoc=N-(9-fluorenylmethoxycarbonyl)-
GC=gas chromatography
Me=methyl
mesyl=methanesulfonyl
PyBroP=bromo-tris(pyrrolidino)phosphonium hexafluorophosphate
rt=room temperature
sat'd=saturated
s-=secondary
t-=tertiary
TFA=trifluoroacetic acid
THF=tetrahydrofuran
TLC=thin layer chromatography

The terms “optional” or “optionally” mean that the subsequently described event or circumstance may or may not occur, and that the description includes instances where the event or circumstance occurs and instances in which it does not. For example, “optionally substituted alkyl” includes “alkyl” and “substituted alkyl,” as defined below. It will be understood by those skilled in the art with respect to any group containing one or more substituents that such groups are not intended to introduce any substitution or substitution patterns that are sterically impractical, synthetically non-feasible, and/or inherently unstable.

The term “alkyl” refers to linear, branched, or cyclic aliphatic hydrocarbon structures and combinations thereof, which structures may be saturated or unsaturated (for example, having up to 20 carbon atoms, such as up to 13 carbon atoms). Lower alkyl refers to alkyl groups having from 1 to 5 (such as 1 to 4) carbon atoms. Examples of lower alkyl groups include methyl, ethyl, propyl, isopropyl, butyl, s- and t-butyl and the like. Alkyl also includes alkanyl, alkenyl and alkynyl residues and therefore includes cyclohexylmethyl, vinyl, allyl, isoprenyl, and the like. Alkylene, alkenylene and alkynylene are other subsets of alkyl, referring to the same residues as alkyl, but having two points of attachment to the parent structure. Examples of alkylene include ethylene (—CH₂CH₂—), ethenylene (—CH═CH—), propylene (—CH₂CH₂CH₂—), dimethylpropylene (—CH₂C(CH₃)₂CH₂—) and cyclohexylpropylene (—CH₂CH₂CH(C₆H₁₃)—). When an alkyl residue having a specific number of carbons is named, all geometric isomers of that residue having the specified number of carbons are included; thus, for example, “butyl” includes n-butyl, sec-butyl, isobutyl and t-butyl; “propyl” includes n-propyl and isopropyl.

The term “cycloalkyl” (or “carbocyclic”) is a subset of alkyl and includes cyclic aliphatic hydrocarbon groups having from 3 to 13 carbon atoms. Examples of cycloalkyl groups include c-propyl, c-butyl, c-pentyl, norbornyl, adamantyl, and the like.

The terms “alkoxy” or “alkoxyl” refer to the group —O-alkyl, for example, including from 1 to 8 carbon atoms in a straight, branched or cyclic configuration, or combinations thereof, attached to the parent structure through an oxygen. Examples include methoxy, ethoxy, propoxy, isopropoxy, cyclopropyloxy, cyclohexyloxy, and the like. Lower-alkoxy refers to groups containing 1 to 5 carbons.

The term “substituted alkoxy” refers to the group —O-(substituted alkyl). One such substituted alkoxy group is “polyalkoxy” or —O-(optionally substituted alkylene)-(optionally substituted alkoxy), and includes groups such as —OCH₂CH₂OCH₃, and glycol ethers such as polyethyleneglycol and —O(CH₂CH₂O)ₓCH₃, where x is an integer from about 2 to 20, such as from 2 to 10 or 2 to 5. Another suitable substituted alkoxy group is hydroxyalkoxy (—OCH₂(CH₂)_{y}OH), where y is an integer from 1 to about 10 or 1 to about 4.

The term “acyl” refers to groups having from 1 to 8 carbon atoms in a straight, branched or cyclic configuration, or combinations thereof attached to the parent structure through a carbonyl functionality, and to a hydrogen atom attached to the parent structure through a carbonyl functionality. Such groups may be saturated or unsaturated, and aliphatic or aromatic. One or more carbons in the acyl residue may be replaced by nitrogen, oxygen or sulfur as long as the point of attachment to the parent remains at the carbonyl. Examples include formyl, acetyl, benzoyl, propionyl, isobutyryl, t-butoxycarbonyl, benzyloxycarbonyl, aminocarbonyl, and the like. Lower-acyl refers to an acyl group containing one to five carbons. “Substituted acyl” refers to an acyl group where one or more of the hydrogens otherwise attached to a carbon, nitrogen or sulfur atom is substituted, the point of attachment to the parent moiety remaining at the carbonyl.

The term “acyloxy” refers to the group —O-acyl. “Substituted acyloxy” refers to the group —O-substituted acyl.

The term “amidino” refers to the group —C(═NH)—NH₂. The term “substituted amidino” refers to the formula —C(═NR)—NRR in which each of the three R groups is independently chosen from hydrogen, optionally substituted alkyl, optionally substituted alkoxy, optionally substituted aminocarbonyl, optionally substituted aryl, optionally substituted heteroaryl, optionally substituted heterocyclyl, acyl, alkoxycarbonyl, sulfanyl, sulfinyl and sulfonyl, provided that at least one R group is not hydrogen.

The term “amino” refers to the group —NH₂. The term “substituted amino” refers to the groups —NHR and —NRR where each of the two R groups is independently chosen from optionally substituted acyl, optionally substituted alkyl, optionally substituted alkoxy, optionally substituted amino, optionally substituted aryl, optionally substituted heteroaryl, optionally substituted heterocyclyl, sulfinyl and sulfonyl, e.g., methylamino, dimethylamino, diethylamino, methylsulfonylamino, furanyl-oxy-sulfonamino, and guanidino.

The terms “aryl” and “heteroaryl” refer to a 5- or 6-membered aromatic ring or heteroaromatic ring, respectively, containing from 1 to 4 heteroatoms selected from O, N, or S; a bicyclic 9- or 10-membered aromatic ring system; a heteroaromatic ring system containing from 1 to 4 (or more) heteroatoms selected from O, N, or S; a tricyclic 13- or 14-membered aromatic ring system; and a heteroaromatic ring system containing from 1 to 4 or more heteroatoms chosen from O, N, and S. Aromatic 6- to 14-membered carbocyclic rings include, e.g., benzene, naphthalene, indane, tetralin, and fluorene and 5- to 10-membered aromatic heterocyclic rings include, e.g., imidazole, imidazoline, pyridine, indole, thiophene, benzopyranone, thiazole, furan, benzimidazole, quinoline, isoquinoline, quinoxaline, pyrimidine, pyrazine, tetrazole and pyrazole; such as imidazole and imidazoline.

The term “aralkyl” refers to a residue in which an aryl moiety is attached to the parent structure via an alkyl residue. Examples include benzyl, phenethyl, phenylvinyl, phenylallyl, and the like. The term “heteroaralkyl” refers to a residue in which a heteroaryl moiety is attached to the parent structure via an alkyl residue. Examples include furanylmethyl, pyridinylmethyl, pyrimidinylethyl, and the like.

The term “aryloxy” refers to the group —O-aryl. Similarly, the terms “aralkoxy” and “heteroaralkoxy” refer, respectively, to an aryl or heteroaryl moiety attached to the parent structure via an alkoxy residue.

The terms “halogen” and “halo” refer to fluorine, chlorine, bromine or iodine (e.g., fluorine, chlorine and bromine). Dihaloaryl, dihaloalkyl, trihaloaryl etc. refer to aryl and alkyl substituted with a plurality of halogens, but not necessarily a plurality of the same halogen; thus 4-chloro-3-fluorophenyl is within the scope of dihaloaryl.

The terms “heterocycle” or “heterocyclyl” refer to a cycloalkyl or aryl residue in which 1 to 4 of the carbons is replaced by a heteroatom such as O, N, or S (i.e., encompassing heterocycloalkyl and heteroaryl). Examples of heterocyclyl residues include imidazolyl, imidazolinyl, pyrrolidinyl, pyrazolyl, pyrrolyl, indolyl, quinolinyl, isoquinolinyl, tetrahydroisoquinolinyl, benzofuranyl, benzodioxanyl, benzodioxolyl (commonly referred to as methylenedioxyphenyl, when occurring as a substituent), tetrazolyl, morpholinyl, thiazolyl, pyridinyl, pyridazinyl, pyrimidinyl, thiophenyl, furanyl, oxazolyl, oxazolinyl, isoxazolyl, dioxanyl, tetrahydrofuranyl, and the like. “N-heterocyclyl” refers to a nitrogen-containing heterocycle as a substituent residue. Examples of N-heterocyclyl residues include 4-morpholinyl, 4-thiomorpholinyl, 1-piperidinyl, 1-pyrrolidinyl, 3-thiazolidinyl, piperazinyl and 4-(3,4-dihydrobenzoxazinyl). Examples of substituted heterocyclyl include 4-methyl-1-piperazinyl and 4-benzyl-1-piperidinyl.

The terms “heteroaryloxy” and “heterocyclooxy” refer to the groups —O-heteroaryl and —O-heterocyclyl, respectively.

The term “solvate” refers to a compound (e.g., a compound of Formula I or a pharmaceutically acceptable salt thereof) in physical association with one or more molecules of a pharmaceutically acceptable solvent.

The term “substituted” as used with regard to alkyl, aryl, aralkyl, heteroaryl and heterocyclyl, refers to an alkyl, aryl, aralkyl, heteroaryl or heterocyclyl moiety wherein one or more (for example, up to about 5, or up to about 3) hydrogen atoms are replaced by a substituent independently chosen from optionally substituted acyl (e.g., aminocarbonyl and alkoxycarbonyl or “esters”), optionally substituted acyloxy (e.g., acid esters, carbamic acid esters, carbonic acid esters, and thiocarbonic acid esters), optionally substituted alkyl (e.g., fluoroalkyl), optionally substituted alkoxy (e.g., methoxy and methoxymethoxy), alkylenedioxy (e.g., methylenedioxy), optionally substituted amino (e.g., alkylamino, dialkylamino, carbonylamino, benzyloxycarbonylamino or “CBZ-amino”, and carboxamido), optionally substituted amidino, optionally substituted aryl (e.g., phenyl and 4-methyl-phenyl or “tolyl”), optionally substituted aralkyl (e.g., benzyl), optionally substituted aryloxy (e.g., phenoxy), optionally substituted aralkoxy (e.g., benzyloxy), optionally substituted heteroaryl, optionally substituted heteroaralkyl, optionally substituted heteroaryloxy, optionally substituted heteroaralkoxy, carboxy (—COOH), cyano, halo, hydroxy, nitro, sulfanyl, sulfinyl, sulfonyl and thio. In the compounds of Formula I wherein T is substituted lower alkylene, the term “substituted” also refers to alkylene groups where one or more (e.g., 1 or up to about 3) carbon atoms are replaced by a heteroatom independently chosen from O, N or S. Thus, the term substituted lower alkylene encompasses —CH₂—S—CH₂—.

The term “sulfanyl” refers to the groups —S-(optionally substituted alkyl), —S-(optionally substituted aryl), —S-(optionally substituted heteroaryl), and —S-(optionally substituted heterocyclyl).

The term “sulfinyl” refers to the groups —S(O)—H, —S(O)-(optionally substituted alkyl), —S(O)-(optionally substituted amino), —S(O)-(optionally substituted aryl), —S(O)-(optionally substituted heteroaryl), and —S(O)-(optionally substituted heterocyclyl).

The term “sulfonyl” refers to the groups —S(O₂)—H, —S(O₂)-(optionally substituted alkyl), —S(O₂)-(optionally substituted amino), —S(O₂)-(optionally substituted aryl), —S(O₂)-(optionally substituted heteroaryl), —S(O₂)-(optionally substituted heterocyclyl), —S(O₂)-(optionally substituted alkoxy), —S(O₂)-optionally substituted aryloxy), —S(O₂)-(optionally substituted heteroaryloxy), and —S(O₂)-(optionally substituted heterocyclyloxy).

The term “isomers” refers to different compounds that have the same molecular formula. The term “stereoisomers” refers to isomers that differ only in the way the atoms are arranged in space. The term “enantiomers” refers to a pair of stereoisomers that are non-superimposable mirror images of each other. The term “racemic” mixture refers to a 1:1 mixture of a pair of enantiomers. The term “(±)” is used to designate a racemic mixture where appropriate. The term “diastereoisomers” refers to stereoisomers that have at least two asymmetric atoms, but which are not mirror-images of each other. Absolute stereochemistry is specified according to the Cahn-Ingold-Prelog R-S system. When a compound is a pure enantiomer the stereochemistry at each chiral carbon may be specified by either (R)- or (S)-. Resolved compounds whose absolute configuration is unknown are designated (+) or (−) depending on the direction (dextro- or levorotatory) which they rotate plane polarized light at the wavelength of the sodium D line. The term “substantially pure” refers to a mixture of compounds having at least about 95% chemical purity with no single impurity greater than about 1%. The terms “substantially optically pure” and “substantially enantiomerically pure” are used interchangeably to refer to mixtures having at least about 95% enantiomeric excess. The invention contemplates the use of pure enantiomers and mixtures of enantiomers, including racemic mixtures, although the use of a substantially optically pure enantiomers is generally most suitable.

The term “mitotic spindle formation” refers to the organization of microtubules into bipolar structures by mitotic kinesins. The term “mitotic spindle dysfunction” refers to mitotic arrest, monopolar spindle formation or mitotic spindle malformation, in which context “malformation” encompasses the splaying of mitotic spindle poles, or otherwise causing morphological perturbation of the mitotic spindle. The term “inhibit”, as used with reference to mitotic spindle formation, means altering mitotic spindle formation, including decreasing spindle formation, and increasing or decreasing spindle pole separation. The term “anti-mitotic” means inhibiting or having the potential to inhibit mitosis, for example, as described above.

The term “composition,” is used interchangeably with the term “formulation”, and refers to a pharmaceutical composition comprising at least one chemical entity chosen from a compound of Formula I and pharmaceutically acceptable salts, solvates, crystal forms, diastereomers, and prodrugs thereof.

The term “pharmaceutically acceptable salt” refers to both acid and base addition salts. The term “pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, or organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. The term “pharmaceutically acceptable base addition salts” include those salts derived from inorganic bases such as sodium, potassium, lithium, ammonium, calcium, magnesium, iron, zinc, copper, manganese, aluminum salts and the like, for example, ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanol amine.

The terms “therapeutically effective amount” and “effective amount” are used interchangeably to refer to that amount of at least one chemical entity chosen from a compound of Formula I, and pharmaceutically acceptable salts, solvates, crystal forms, diastereomers, and prodrugs thereof, that is sufficient to effect treatment, as defined below, when administered to a patient in need of such treatment. The effective amount will vary depending upon the patient and disease condition being treated, the weight and age of the patient, the severity of the disease condition, the particular chemical entity chosen, the dosing regimen to be followed, timing of administration, the manner of administration, and the like, all of which can readily be determined by one of ordinary skill in the art. In one aspect of the invention, the effective amount will be the amount of at least one chemical entity chosen from a compound of Formula I, and pharmaceutically acceptable salts, solvates, crystal forms, diastereomers, and prodrugs thereof, sufficient to inhibit KSP kinesin activity in cells involved with the disease being treated.

The terms “treatment” and “treating” refer to any treatment of a disease in a patient, including:
a) preventing the disease, that is, causing the clinical symptoms of the disease not to develop;
b) inhibiting the disease, that is, slowing or arresting the development of clinical symptoms; and/or
c) relieving the disease, that is, causing the regression of clinical symptoms.

The term “patient” includes humans and other animals, particularly mammals, and other organisms. Thus, the methods are applicable to both human therapy and veterinary applications. In one embodiment the patient is a mammal, most particularly the patient is human.

The present invention provides certain heterocyclic-fused pyrimidinone derivative compounds. The compounds are inhibitors of one or more mitotic kinesins. By inhibiting mitotic kinesins, such as KSP, but not other kinesins (e.g., transport kinesins), specific inhibition of cellular proliferation is accomplished. Thus, the present invention capitalizes on the finding that perturbation of mitotic kinesin function causes malformation or dysfunction of mitotic spindles, frequently resulting in cell cycle arrest and cell death.

Accordingly, the present invention relates to at least one chemical entity chosen from a compound of Formula I: 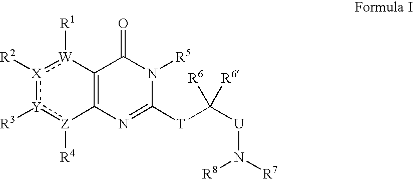
and pharmaceutically acceptable salts, solvates, crystal forms, diastereomers, and prodrugs thereof, wherein:
T and U are independently absent or optionally substituted lower alkylene;
W, X and Y are independently chosen from —N═, —N—, —C═, CH, CRⁱ, O and S;
Z is chosen from —N═, —N—, —C═, CH, CRⁱ, O and S, or is absent, provided that:
   no more than two of W, X, Y and Z are both —N═, O or S, and
   W, X, Y or Z can be O or S only when the ring they form is not aromatic, or W, X or Y can be O or S when Z is absent;
Rⁱ is chosen from optionally substituted alkyl, optionally substituted alkoxy, optionally substituted aryl, optionally substituted heteroaryl, halo and cyano;
R¹, R², R³ and R⁴ are independently chosen from hydrogen, hydroxy, optionally substituted alkyl (including, but not limited to, fluoroalkyl, sulfonamidoalkyl and carboxyalkyl), optionally substituted alkoxy, alkylsulfonyl, alkylsulfonamido, carboxamido, alkylthio, aminocarbonyl, optionally substituted aryl, optionally substituted heteroaryl, halo, nitro and cyano, provided that R¹, R² or R³ is absent when W, X or Y, respectively, is —N═, S, or O, and R⁴ is absent when Z is —N═, S, or O or is absent;
R⁵ is chosen from optionally substituted alkyl, optionally substituted aryl and optionally substituted heteroaryl;
R⁶ is chosen from optionally substituted alkyl and optionally substituted aryl,
R^{6′} is chosen from hydrogen, optionally substituted alkyl and optionally substituted aryl;
R⁷ is chosen from optionally substituted alkyl, optionally substituted aryl and optionally substituted heteroaryl; and
R⁸ is chosen from hydrogen, —C(O)—R⁹, —S(O)₂—R^{9′}, —CH₂—R⁹, —C(O)—O—R^{9′}, —C(O)—NH—R⁹ or —S(O)₂—NH—R⁹, wherein:
   R⁹ is chosen from hydrogen, optionally substituted alkyl, optionally substituted aryl and optionally substituted heteroaryl, and
   R^{9′} is chosen from optionally substituted alkyl, optionally substituted aryl and optionally substituted heteroaryl;
or alternatively:
R⁶ and R^{6′}, taken together with the carbon to which they are bonded, form an optionally substituted cycloalkyl or optionally substituted heterocycloalkyl having 5 to 7 ring atoms; or
R⁶ and R⁷ taken together with the nitrogen to which R⁷ is bonded form an optionally substituted 5- to 12-membered heterocycle having up to 2 additional heteroatoms selected from O, N and S, provided that such heterocycle is not optionally substituted: 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl; or
R⁷ and R⁸, taken together with the nitrogen to which they are bonded form an optionally substituted 5- to 12-membered heterocycle having up to 2 additional heteroatoms selected from O, N and S, provided that such heterocycle is not optionally substituted: I-piperazin-1-yl, 1-[1,4]diazepan-1-yl, 2-oxo-[1,4]diazepan-1-yl, 7-oxo-[1,4]diazepan-1-yl, imidazol-1-yl or imidazolin-1-yl; or
R⁶ taken together with R⁷, and R^{6′} taken together with R⁸ and the nitrogen to which R⁷ and R⁸ are bonded form an optionally substituted 10- to 13-membered fused heterocycle having up to 2 additional heteroatoms selected from O, N and S,
   provided that:
at least one of W, X, Y or Z is other than —C═; and
when T is a covalent bond, then Z is present and at least one of W, X, Y or Z is O or S; and
when T is a covalent bond and R⁸ is hydrogen, —C(O)—R⁹, —S(O)₂—R^{9′}, —CH₂—R⁹, —C(O)—O—R^{9′}, —C(O)—NH—R⁹ or —S(O)₂—NH—R⁹, and when R⁷ and R⁸ are optionally substituted imidazolyl or optionally substituted imidazolinyl, then at least one of W, X, Y or Z is —N—, CH, CRⁱ, O or S.
   The dashed lines in Formula I indicate that the corresponding bond may be a single bond (e.g., where X is CH) or a double bond (e.g., where X is —C═).

In one of its particular aspects, the present invention pertains to compounds, methods and compositions employing at least one chemical entity chosen from a compound of Formula I and pharmaceutically acceptable salts, solvates, crystal forms, diastereomers, and prodrugs thereof, where:
T is optionally substituted lower alkylene;
U is chosen from a covalent bond or optionally substituted lower alkylene;
W, X and Y are independently chosen from —N═, N, —C═, CH, CRⁱ, O and S;
Z is chosen from —N═, N, —C═, CH, CRⁱ, O and S, or is absent, provided that:
   no more than two of W, X, Y and Z are both —N═, O or S, and
   W, X, Y or Z can be O or S only when the ring they form is not aromatic, or W, X or Y can be O or S when Z is absent;
Rⁱ is chosen from optionally substituted alkyl, optionally substituted alkoxy, optionally substituted aryl, optionally substituted heteroaryl, halogen and cyano;
R¹, R², R³ and R⁴ are independently chosen from hydrogen, hydroxy, optionally substituted alkyl (including, but not limited to, fluoroalkyl, sulfonamidoalkyl and carboxyalkyl), optionally substituted alkoxy, alkylsulfonyl, alkylsulfonamido, carboxamido, alkylthio, aminocarbonyl, optionally substituted aryl (including, but not limited to, sulfonamidoaryl), optionally substituted heteroaryl, halogen, nitro and cyano, provided that R¹, R² or R³ is absent where W, X or Y, respectively, is —N═, S, or O, and R⁴ is absent where Z is —N═, S, O or is absent;
R⁵ is chosen from optionally substituted alkyl, optionally substituted aryl and optionally substituted heteroaryl;
R⁶ is chosen from optionally substituted alkyl and optionally substituted aryl,
R^{6′} is chosen from hydrogen, optionally substituted alkyl and optionally substituted aryl;
R⁷ is chosen from optionally substituted alkyl, optionally substituted aryl and optionally substituted heteroaryl; and
R⁸ is chosen from hydrogen, —C(O)—R⁹, —S(O)₂—R^{9′}, —CH₂—R⁹, —C(O)—O—R^{9′}, —C(O)—NH—R⁹ or —S(O)₂—NH—R⁹, in which:
   R⁹ is chosen from hydrogen, optionally substituted alkyl, optionally substituted aryl and optionally substituted heteroaryl, and
   R^{9′} is chosen from optionally substituted alkyl, optionally substituted aryl and optionally substituted heteroaryl;
or alternatively:
R⁶ taken together with R^{6′} is optionally substituted cycloalkyl or optionally substituted heterocycloalkyl having 5 to 7 ring atoms;
R⁶ and R⁷ taken together with the nitrogen to which R⁷ is bonded form an optionally substituted 5 to 12 membered heterocycle having up to 2 additional heteroatoms selected from O, N and S; or
R⁷ and R⁸, taken together with the nitrogen to which they are bonded form an optionally substituted 5 to 12 membered heterocycle having up to 2 additional heteroatoms selected from O, N and S; or
R⁶ taken together with R⁷, and R^{6′} taken together with R⁸ and the nitrogen to which R⁷ and R⁸ are bonded form an optionally substituted 10 to 13-membered fused heterocycle having up to 2 additional heteroatoms selected from O, N and S.

In one of its particular aspects, the present invention pertains to compounds, methods and compositions employing at least one chemical entity chosen from a compound of Formula I and pharmaceutically acceptable salts, solvates, crystal forms, diastereomers, and prodrugs thereof, wherein T is optionally substituted lower alkylene, further having one or more of the following:
X, Y or Z is —N═, or W and Z are —N═;
R¹, R², R³ and R⁴ are independently hydrogen, halo (particularly chloro and fluoro), lower alkyl (particularly methyl), substituted lower alkyl, lower alkoxy (particularly methoxy), cyano or absent; more particularly three of R¹, R², R³ and R⁴ are hydrogen, or two of R¹, R², R³ and R⁴ are hydrogen and a third is halo, methoxy or cyano, and the fourth is absent;
R⁵ is other than optionally substituted phenyl (particularly optionally substituted aralkyl, such as benzyl or substituted benzyl; more particularly benzyl);
R⁶ is lower alkyl (particularly methyl, ethyl, i-propyl, c-propyl or t-butyl);
R^{6′} is hydrogen;
R⁷ is substituted alkyl (particularly primary-amino-substituted lower alkyl, secondary-amino-substituted lower alkyl or tertiary-amino-substituted lower alkyl); more particularly 3-amino-propyl; and/or
R⁸ is —C(O)—R⁹, in which R⁹ is optionally substituted alkyl (particularly lower alkyl, haloalkyl, and lower alkoxyalkyl), optionally substituted aryl (particularly phenyl, lower alkyl-, lower alkoxy-, and/or halo-substituted phenyl), optionally substituted aralkyl (particularly optionally substituted benzyl and phenylvinyl), aryloxyalkyl (particularly phenoxy lower alkyl), optionally substituted heteroaryl, optionally substituted heteroaralkyl or optionally substituted heteroaryloxyalkyl; more particularly where R⁹ is methoxy-methyl or p-tolyl;
R⁸ is —C(O)—OR^{9′}, in which R^{9′} is: optionally substituted aryl (particularly phenyl, lower alkyl-, lower alkoxy-, and/or halo-substituted phenyl) or optionally substituted heteroaryl;
R⁶ and R⁷ taken together with the nitrogen to which R⁷ is bonded form an optionally substituted 5 to 7 (particularly 5 or 6) membered heterocycle having up to 2 additional heteroatoms selected from O, N and S (particularly optionally substituted 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl); and
R⁷ and R⁸ taken together with the nitrogen to which they are bonded form an optionally substituted 5 to 7 (particularly 5 or 6) membered heterocycle having up to 2 additional heteroatoms selected from O, N and S (particularly optionally substituted: 1-piperazin-1-yl, 1-[1,4]diazepan-1-yl, imidazol-1-yl or imidazolin-1-yl).

In another of its particular aspects, the present invention pertains to compounds, methods and compositions employing at least one chemical entity chosen from a compound of Formula I and pharmaceutically acceptable salts, solvates, crystal forms, diastereomers, and prodrugs thereof, wherein R⁵ is other than optionally substituted phenyl when R⁶ is methyl, optionally having one or more of the following:
R⁸ is —S(O)₂—R^{9′}, in which R^{9′} is: C₁-C₁₃ alkyl; heteroaryl; naphthyl; or phenyl optionally substituted with halo, lower alkyl, lower alkoxy, nitro, methylenedioxy, phenyl or trifluoromethyl; or
R⁸ is —CH₂—R⁹, in which R⁹ is: C₁-C₁₃ alkyl; substituted lower alkyl; benzyl; heterocyclyl; naphthyl; or phenyl optionally substituted with halo, lower alkyl, lower alkoxy, nitro, methylenedioxy, phenyl or trifluoromethyl; or
R⁷ taken together with R⁸ is 2-(optionally substituted)-4,4-(optionally di-substituted)-4,5-dihydro-imidazol-1-yl, 2-(optionally substituted phenyl)-imidazol-1-yl, or 4-(optionally substituted alkyl)-2-(optionally substituted aryl)-imidazol-1-yl
   optionally having one or more of the following:
X, Y or Z is —N═;
R¹, R², R³ and R⁴ are independently hydrogen, chloro, fluoro, lower alkyl substituted lower alkyl, methoxy, cyano or absent;
R⁵ is benzyl or substituted benzyl;
R⁶ is ethyl, i-propyl, c-propyl or t-butyl; and/or
R⁷ is a primary-amino-substituted lower alkyl, secondary-amino-substituted lower alkyl or tertiary-amino-substituted lower alkyl;
   and particularly where:
R⁹ is heterocyclyl; naphthyl; or phenyl substituted with halo, lower alkyl, lower alkoxy, nitro, methylenedioxy, phenyl or trifluoromethyl; or
R⁷ taken together with R⁸ is 2-(4-methylphenyl)-4,5-dihydro-imidazol-1-yl, 2-(3-fluoro-,4-methylphenyl)-4,5-dihydro-imidazol-1-yl, 2-(4-methylphenyl)-4,4-dimethyl-4,5-dihydro-imidazol-1-yl, 2-(3-fluoro-,4-methylphenyl)-4,4-dimethyl-4,5-dihydro-imidazol-1-yl, 2-phenyl-imidazol-1-yl, 2-p-toluoyl-imidazol-1-yl, 2-(4-fluorophenyl)-imidazol-1-yl, 2-(4-chlorophenyl)-imidazol-1-yl, 2-(3-fluoro-4-methylphenyl)-imidazol-1-yl, 4-(2-amino-ethyl)-2-phenyl-imidazol-1-yl, 4-(2-amino-ethyl)-2-p-tolyl-imidazol-1-yl, 4-(2-amino-ethyl)-2-(4-fluoro-phenyl)-imidazol-1-yl, 4-(2-amino-ethyl)-2-(4-chloro-phenyl)-imidazol-1-yl, 4-(2-amino-ethyl)-2-(3-fluoro-4-methyl-phenyl)-imidazol-1-yl, 4-(aminomethyl)-2-phenyl-imidazol-1-yl, 4-(aminomethyl)-2-p-tolyl-imidazol-1-yl, 4-(aminomethyl)-2-(4-fluoro-phenyl)-imidazol-1-yl, 4-(aminomethyl)-2-(4-chloro-phenyl)-imidazol-1-yl, or 4-(aminomethyl)-2-(3-fluoro-4-methyl-phenyl)-imidazol-1-yl.

In still another of its particular aspects, the present invention pertains to compounds, methods and compositions employing at least one chemical entity chosen from a compound of Formula I and pharmaceutically acceptable salts, solvates, crystal forms, diastereomers, and prodrugs thereof, of the (R)-configuration.

Many of the compounds described herein contain one or more asymmetric centers (e.g., the carbon to which R⁶ and R⁶′ are attached when R⁶ and R⁶′ are different) and may thus give rise to enantiomers, diastereomers, and other stereoisomeric forms that may be defined, in terms of absolute stereochemistry, as (R)- or (S)-. When the compounds described herein contain olefinic double bonds or other centers of geometric asymmetry, and unless specified otherwise, it is intended that such compounds include both E and Z geometric isomers. All tautomeric forms are also intended to be included. The present invention is meant to include all such possible isomers, including racemic mixtures, intermediate mixtures, optically pure forms, substantially optically pure forms, substantially enantiomerically pure forms, and substantially pure forms, for example, in the particular embodiment where R⁶ and R⁶′ are different and the stereogenic center to which R⁶ is attached has an (R)-configuration.

The compounds of Formula I can be named and numbered (e.g., using AutoNom version 2.1 in ISIS-DRAW or CS ChemDraw Ultra®, Cambridgesoft Corp., Cambridge, Mass.) as described below. For example, the compound of Formula IA: 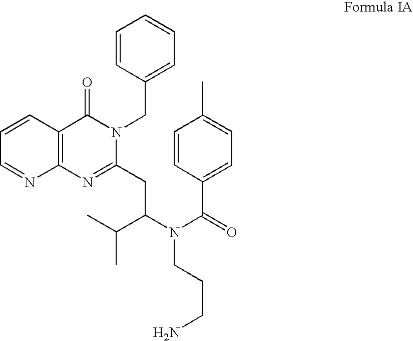
i.e., the compound of Formula I where T is methylene; U is a covalent bond; W, X and Y are —C═; Z is —N═; R¹, R² and R³ are H; R⁴ is absent; R⁵ is benzyl; R⁶ is i-propyl; R^{6′} is H; R⁷ is amino-propyl-; and R⁸ is —C(O)—R⁹ where R⁹ is p-methyl-phenyl, can be named N-(3-amino-propyl)-N-[1-(3-benzyl-4-oxo-3,4-dihydro-pyrido[2,3-d]pyrimidin-2-ylmethyl)-2-methyl-propyl]-4-methyl-benzamide.

The compound of Formula IB: 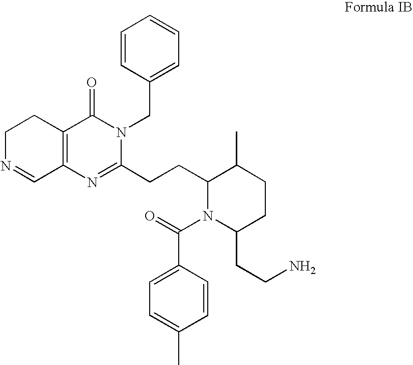
i.e., the compound of Formula I where T is n-propylene; U is a covalent bond; W and X are CH; Y is —N═; Z is —C═; R¹, R² and R⁴ are H; R³ is absent; R⁵ is benzyl; R^{6′} is H; R⁶ and R⁷ taken together with the nitrogen to which R⁷ is bound form a 6-(2-amino-ethyl)-3-methyl-piperidin-2-yl moiety; and R⁸ is —C(O)—R⁹ where R⁹ is p-methyl-phenyl, can be named 2-{2-[6-(2-amino-ethyl)-3-methyl-1-(4-methyl-benzoyl)-piperidin-2-yl]-ethyl}-3-benzyl-5,6-dihydro-3H-pyrido[3,4-d]pyrimidin-4-one.

The compound of Formula IC: 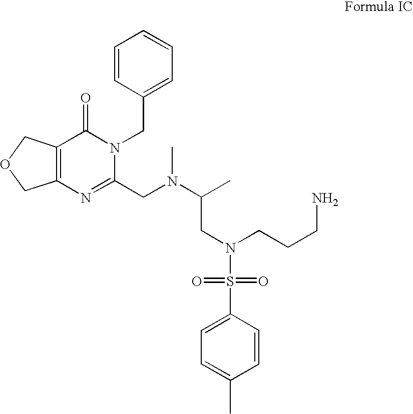
i.e., the compound of Formula I where T is 2-methyl-aminomethylene; U is methylene; W and Y are CH; X is O; Z is absent; R¹ and R³ are H; R² and R⁴ are absent; R⁵ is benzyl; R⁶ is methyl; R^{6′} is H; R⁷ is amino-propyl-; and R⁸ is —S(O)₂—R^{9′} where R^{9′} is p-methyl-phenyl, can be named N-(3-amino-propyl)-N-{2-[(3-benzyl-4-oxo-3,4,5,7-tetrahydro-furo[3,4-d]pyrimidin-2-ylmethyl)-methyl-amino]-propyl}-4-methyl-benzenesulfonamide.

The compound of Formula ID: 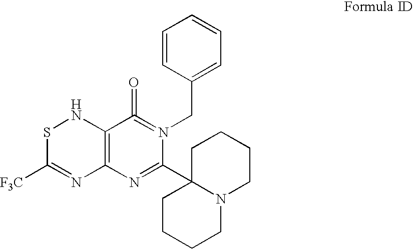
i.e., the compound of Formula I where T and U are each a covalent bond; W is N; X is S; Y is —C═; and Z is —N═; R¹ is H; R² and R⁴ are absent; R³ is trifluoromethyl; R⁵ is benzyl; R⁶ taken together with R⁷, and R^{6′} taken together with R⁸ and the nitrogen to which R⁷ and R⁸ are bonded form an octahydro-quinolizin-9a-yl heterocycle; can be named 7-benzyl-6-(octahydro-quinolizin-9a-yl)-3-trifluoromethyl-1,7-dihydro-2-thia-1,4,5,7-tetraaza-naphthalen-8-one.

The terms “solvent”, “inert organic solvent,” and “inert solvent” mean a solvent inert under the conditions of the reaction being described in conjunction therewith. Such solvents include, for example, benzene, toluene, acetonitrile, tetrahydrofuran (“THF”), dimethylformamide (“DMF”), chloroform, methylene chloride (or dichloromethane), diethyl ether, methanol, pyridine and the like. Unless specified to the contrary, the solvents used in the reactions of the present invention are inert organic solvents.

Isolation and purification of the compounds and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples hereinbelow. However, other equivalent separation or isolation procedures can, of course, also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. For example, a compound of Formula I can be dissolved in a lower alkanol and placed on a Chiralpak AD (205×20 mm) column (Chiral Technologies, Inc.) conditioned for 60 min at 70% EtOAc in Hexane. It will be appreciated that where the desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents, or by converting one enantiomer to the other by asymmetric transformation.

The compounds of Formula I can be synthesized, for example, as described in PCT Publication Nos. WO 01/30768 and WO 01/98278 or in one or more of co-pending applications: U.S. Application Publication Nos. 2004/0067969 A1, 2004/0116438 A1, 2004/0142949 A1, and 2004/0048853 A1; and PCT Publication No. WO 2004/034972 A2, and/or as described below with reference to Reaction Schemes 1 to 3.

The optionally compounds of Formulae 101, 201, 301 and other reactants are commercially available, e.g., from Aldrich Chemical Company, Milwaukee, Wis. or may be readily prepared by those skilled in the art using commonly employed synthetic methodology.

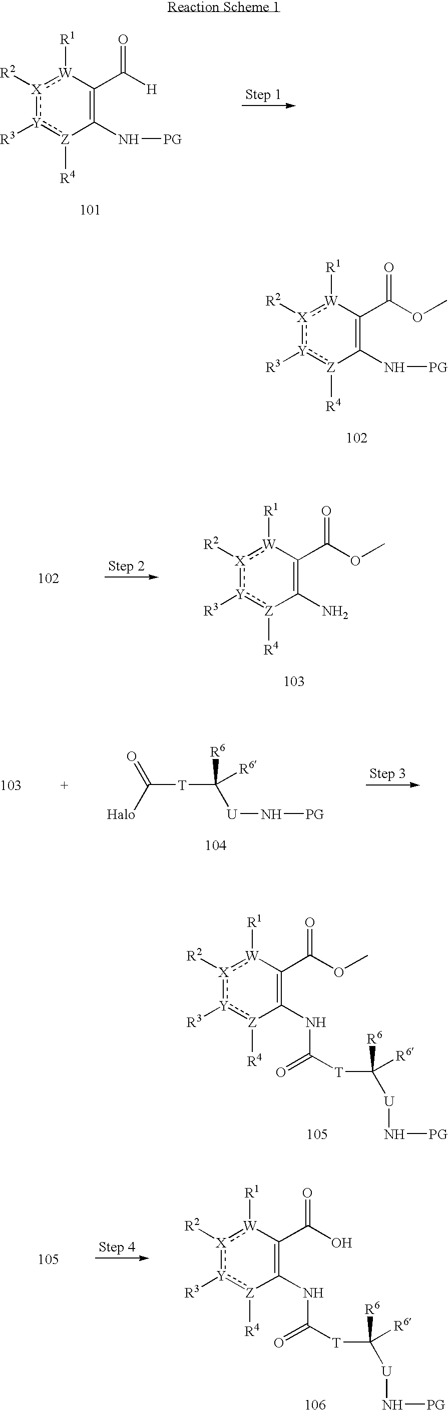

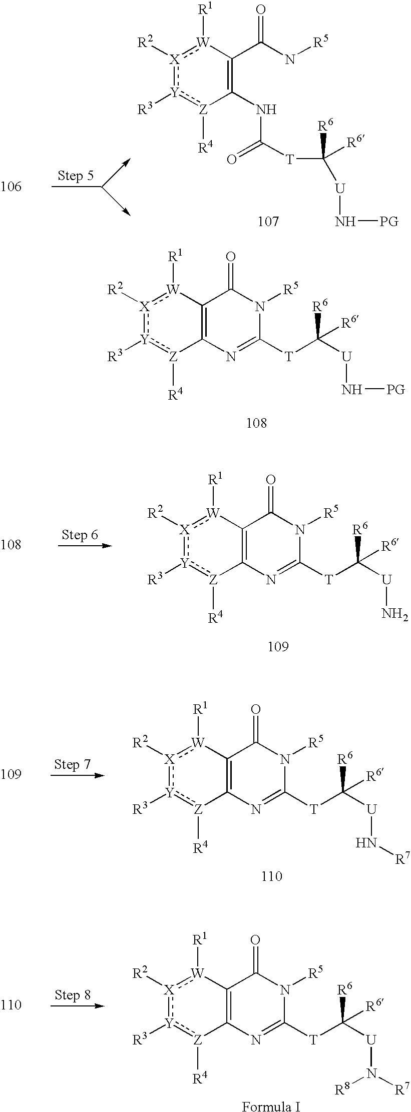

Preparation of Formula 102 Referring to Reaction Scheme 1, Step 1, a protected 4-amino-(hetero)cyclyl carboxaldehyde of Formula 101 (where “PG” is a protecting group such as Fmoc or Boc) is dissolved in a solvent (e.g., methanol) and contacted with a molar excesses of N-iodosuccinimide and potassium carbonate. The reaction takes place with stirring at room temperature over a period of 1 hour, followed by quenching with saturated Na₂S₂O₃. The corresponding methyl ester of Formula 102 is then isolated and purified.

Preparation of Formula 103 Referring to Reaction Scheme 1, Step 2, a methyl ester of Formula 102 is de-protected, for example using TFA (e.g., dissolved in 50/50 solution of TFA/DCM). The reaction takes place with stirring at room temperature over a period of 2 hours. Following conventional work-up, the corresponding de-protected methyl ester of Formula 103 is obtained.

Preparation of Formula 105 For the sake of brevity in the remaining description of the syntheses of compounds of Formula I, it should be understood that either single isomer or a mixture of isomers may be employed to give the corresponding product. In that regard, while the R⁶ substituent in Formula 105 is illustrated as a single enantiomer, the reaction steps should not be taken to be so-limited, and are meant to encompass either single isomer or any mixture of isomers. The compounds where R⁶ taken together with R^{6′} is optionally substituted cycloalkyl or optionally substituted heterocycloalkyl having 5 to 7 ring atoms can be prepared by use of the corresponding acid halide of Formula 104.

Referring to Reaction Scheme 1, Step 3, a de-protected methyl ester of Formula 103 is dissolved in a solvent (e.g., dichloromethane) with a slight molar excess of diisopropylethyl amine. A slight molar excess of an acid halide of Formula 104 (prepared, for example by contacting an N-protected carbonyl amino acid, such as a chiral amino acid, with a cyanuric halide) is added. The reaction takes place with stirring at room temperature over a period of 16 hours. Solvent removal under reduced pressure and purification by flash silica gel chromatography afford the corresponding amide of Formula 105.

Preparation of Formula 106 Referring to Reaction Scheme 1, Step 4, a compound of Formula 105 is dissolved in a solvent and contacted with a twice molar excess of sodium hydroxide. The reaction takes place with stirring at room termperature over a period of 1 hour, followed by the addition of silica gel with continued stirring for an additional 10 minutes. The silica gel is filtered off and the solvents evaporated to afford the corresponding carboxylic acid of Formula 106.

Preparation of Formulae 107 and 108 Referring to Reaction Scheme 1, Step 5, an acid of Formula 106 is dissolved (e.g., in dichloromethane) and contacted with a slight molar excess of a coupling reagent such as EDC. The solution is stirred at room temperature for 2 hours until the complete consumption of the starting material is observed by reverse phase HPLC. A twice molar excess of a primary amine of the formula R⁵—NH₂ (such as benzylamine) is added. The reaction takes place with stirring at room temperature over a period of 16 hours. The mixture is concentrated under vacuum and purified by silica gel chromatography to separate the uncyclized product of Formula 107 and the cyclized, enantiomerically pure product of Formula 108.

Preparation of Formula 109 Referring to Reaction Scheme 1, Step 6, an enantiomerically pure, cyclized compound of Formula 108 where PG is Cbz is de-protected, for example by dissolution in a solution of HBr/acetic acid. The reaction takes place with stirring at room temperature over a period of 2 hours. Solvent removal affords the corresponding enantiomerically pure de-protected amine of Formula 109, which is optionally isolated and purified.

Preparation of Formula 110 Referring to Reaction Scheme 1, Step 7, a compound of Formula 109 is dissolved in an inert solvent (such as dichloromethane) in the presence of a slight molar excess of an alkali metal acetate borohydride [such as Na(OAc)₃BH] and the solution cooled to 10° C. To this solution is added, portionwise, 1.4 molar equivalents of an aldehyde comprising R⁷ [i.e., a compound having formula R⁷—CHO where R⁷ is as described above or is a protected precursor to such a substituent, e.g., (3-oxo-propyl)-carbamic acid tert-butyl ester or 3-(1,3-dioxo-1,3-dihydro-isoindol-2-yl)-propionaldehyde]; the solution can be allowed to warm to room temperature. Completion over a period of 3 hours is monitored, e.g., by TLC. The corresponding, optionally substituted compound of Formula 110 is isolated and purified.

The protected R⁷ substituent can be de-protected at this point (e.g., when PG is Boc, by reaction with a molar excess of TFA) to give the corresponding compound of Formula 110, which is also a compound of Formula I where R⁸ is H. Alternatively, the compound of Formula 110 (whether R⁷-protected or not, as appropriate) can be carried forward as described below and further derivatized to give the compounds of Formula I where R⁸ is other than H.

Preparation of Formula I where R⁸ is —C(O)—R⁹—S(O)ₙ—R^{9′}, —CH₂—R⁹, —C(O)—O—R⁹, —C(O)—NH—R⁹ or —S(O)₂—NH—R⁹ Referring to Reaction Scheme 1, Step 8, a compound of Formula 110 and an inert solvent (such as dichloromethane) in the presence of over 2 molar equivalents of DIPEA is stirred until dissolved. To this solution is added a slight molar excess of an R⁸ halide [such as, Cl—C(O)—R⁹, Cl—S(O)₂—R^{9′}, Cl—CH₂—R⁹, Cl—C(O)—O—R^{9′} and Cl—S(O)₂—NH—R⁹] or an R⁸ isocyanate (such as O═C═N—R⁹) or an anhydride (such as O[C(O)R^{9′}]₂ or O[S(O)₂R^{9′}]₂) where R⁹ and R^{9′} are as described above, e.g., toluoyl chloride. The reaction takes place, with stirring, at room temperature to 50° C. over a period of 4 to 6 hours. Completion is monitored, e.g., by TLC. The corresponding compound of Formula I is de-protected (if necessary) isolated and purified.

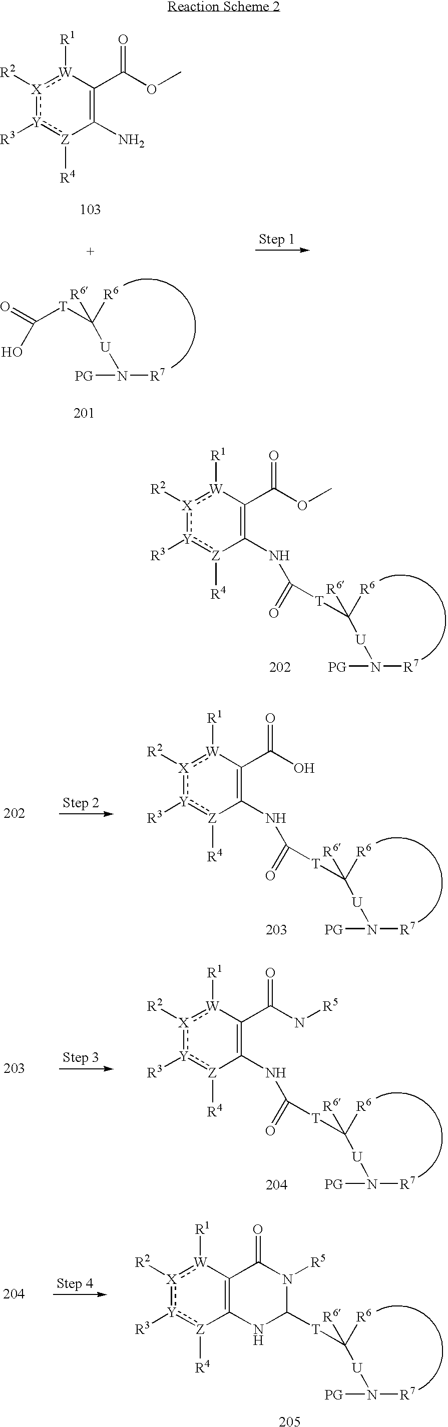

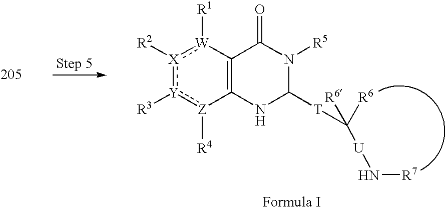

Referring to Reaction Scheme 2, R⁶ and R⁷ are illustrated as combining to form an optionally substituted heterocycle having from 5 to 12 ring atoms including up to 2 additional heteroatoms (not shown) selected from O, N and S. Also not shown are the optional substituents, which can be pendant from from any available ring atom. In that regard, those skilled in the art will appreciate that certain substituents will require protection (typically entailing the addition of a protecting group “PG” to such substituent in the compound of Formula 201 prior to Step 1 of Reaction Scheme 2, for example, as illustrated with regard to the existing nitrogen of the heterocycle) followed by deprotection (typically after the cyclization in Step 4). Compounds of Formula I where such substituent is aryl or heteroaryl can be obtained by starting with the corresponding arylated compound of Formula 201 or by a palladium catalyzed arylation (e.g., as described by Wolfe, J. P.; Tomori, H; Sadighi, J. P.; Yin, J.; and Buchwald, S. L., J. Org. Chem., 2000, 65, 1158-1174) of such a de-protected product.

Preparation of Formula 202 Referring to Reaction Scheme 2, Step 1, a solution is made of an optionally substituted o-amino alicyclic, heterocyclic or (hetero)aryl acid of Formula 103 (prepared, for example, as described with reference to Reaction Scheme 1), a slight molar excess of an N-protected heterocyclic compound of Formula 201 and a slight molar excess of PyBroP dissolved in an organic solvent (e.g., diisopropylethylamine in pyridine). The solution is stirred for 12 to 20 hours at room temperature to afford the corresponding compound of Formula 202, which is conventionally isolated and purified.

Preparation of Formula 203 Referring to Reaction Scheme 2, Step 2, a compound of Formula 202 is dissolved in an organic solvent (e.g., a lower alkanol such as ethanol) and treated with an aqueous hydroxide (e.g., NaOH aq. solution). The mixture is stirred at room temperature for 12 to 20 hours and then concentrated under vacuum. The residue is washed (e.g., with saturated NaCl and concentrated phosphoric acid) and then extracted (e.g., with dichloromethane). Conventional isolation and purification affords the corresponding acid of Formula 203.

Preparation of Formula 204 Referring to Reaction Scheme 2, Step 3, to a solution of Formula 203 in an organic solvent (e.g., dichloromethane) is added molar excesses of EDC and diisopropylethylamine. After stirring at room temperature for 1 to 2 hours, a molar excess of an amine of the formula H₂N—R⁵ (such as benzylamine) is added and the reaction is stirred for an additional 24 to 60 hours. The mixture is washed, dried and isolated to afford the corresponding di-carbamoyl compound of Formula 204, which is taken forward without further purification (this reaction product may also include a small amount of the corresponding cyclized compound of Formula 205).

Preparation of Formula 205 Referring to Reaction Scheme 2, Step 4, a compound of Formula 204 (optionally in the presence of a compound of Formula 205) is stirred in ethylene glycol with K₂CO₃ at 120° C. for 12 to 20 hours, allowed to cool to room temperature, and is extracted (e.g., with dichloromethane). The organic fractions are conventionally isolated and purified to afford the pure cyclized compound of Formula 205.

Preparation of Formula I where the Ring Nitrogen is Not Substituted Referring to Reaction Scheme 2, Step 5, a solution of Formula 205 where PG is a Boc group, is treated with a 1:1 mixture of TFA/dichloromethane for 30 minutes to 2 hours at room temperature. The solution is concentrated under vacuum and partitioned (e.g., between dichloromethane and saturated NaHCO₃). The aqueous layer is extracted (e.g., with dichloromethane) and the combined organic layers are conventionally isolated to afford the corresponding de-protected heterocyclic product of Formula I, which can be purified or taken forward without further purification.

Preparation of Formula I where the Ring Nitrogen is Acylated To a solution of Formula I (obtained, e.g., as described with reference to Reaction Scheme 2, Step 5) in an organic solvent (e.g., dichloromethane) is added molar excesses of an acyl halide (e.g., toluoyl chloride) and diisopropylethylamine. The mixture is stirred for 30 minutes to 2 hours and then partitioned (e.g., between saturated NaHCO₃ and ethyl acetate). Conventional isolation and purification of the organic phase affords the corresponding N-acyl compound of Formula I.

Preparation of Formula I where the Ring Nitrogen is Optionally Substituted with Alkyl, Optionally Substituted with Aralkyl or Optionally Substituted with Heteroaralkyl To a solution of Formula I (obtained, e.g., as described with reference to Reaction Scheme 2, Step 5) in an organic solvent (e.g., dichloromethane) is added a slight molar excess of an aldehyde comprising the desired substituent (e.g., as employed in Reaction Scheme 1, Step 8) and a molar excess of NaHB(OAc)₃. The mixture is stirred for 30 minutes to 1 hour at room temperature. The reaction is quenched with saturated NaHCO₃ and the aqueous phase extracted (e.g., with dichloromethane). Isolation and purification of the organic phase affords the corresponding N-optionally substituted-alkyl, -aralkyl or -heteroaralkyl compound of Formula I.

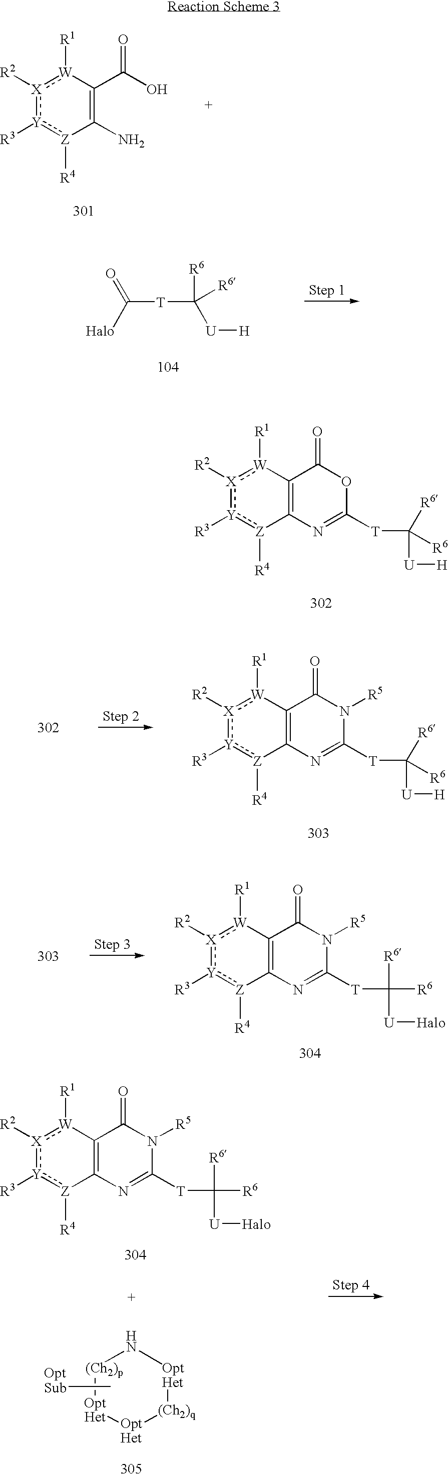

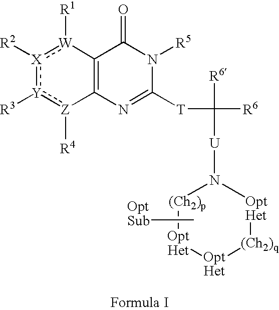

Preparation of Formula 302 Referring to Reaction Scheme 3, Step 1, to an optionally substituted amino-(hetero)cyclic acid of Formula 301 dissolved in an inert organic solvent (such as THF) in the presence of sodium bicarbonate and a dehydrating agent (such as Na₂SO₄) is added a slight molar excess of an optionally substituted acid halide of Formula 104, maintaining room temperature. Completion of the reaction takes place over 2 hours and is monitored, e.g., by TLC. The solvent is then replaced with acetic anhydride, which is heated to 90-100° C. for about 16 hours, monitoring completion of the reaction (e.g., by TLC) followed by removal of the acetic anhydride under vacuum at 80-100° C. The reaction mixture is cooled and the corresponding, cyclized compound of Formula 302 is isolated and purified.

Preparation of Formula 303 Referring to Reaction Scheme 3, Step 2, about 1.5 molar equivalents of a primary amine of the formula R⁵—NH₂ where R⁵ is as described above (such as benzylamine) and 1 molar equivalent of a compound of Formula 302 in an inert organic solvent (such as toluene or chloroform) are heated to reflux. The reaction takes place over a period of 1 to 5 hours, e.g., 3 hours. After removal of water, ethylene glycol and sodium hydroxide (or sodium carbonate) are added to the reaction mixture and the temperature raised to 110-120° C. Completion of the reaction is monitored, e.g., by TLC. The corresponding compound of Formula 303 is isolated and purified.

Preparation of Formula 304 Referring to Reaction Scheme 3, Step 3, a compound of Formula 303, dissolved in acetic acid and in the presence of sodium acetate, is heated to 30-50° C., followed by the addition (with agitation) of a slight molar excess of a halogen (e.g., bromine) in acetic acid over a period of 2 to 4 hours. Completion is monitored, e.g., by TLC; if the starting material continues to be present, the temperature is increased to 50° C. until completion. The corresponding halide of Formula 304 is isolated and purified.

Preparation of Formula I Referring to Reaction Scheme 3, Step 4, an optionally substituted 2-halo-alkyl-compound of Formula 304, one-half molar equivalent of an optionally substituted heterocycle of Formula 305 (where p and q are independently integers from 0 to 8, provided that p+q=1 to 8, up to two of the optional heteroatoms (Opt Het's) can be a heteroatom selected from N, O and S, and an optional substituent (Opt Sub) may be at any permissible position on the heterocycl, including a protecting group, “PG,” such as Boc, as appropriate) and an excess of potassium carbonate are combined in an organic solvent (e.g., acetonitrile). The reaction takes place under a nitrogen atmosphere at elevated temperature (e.g., 100° C.) over a period of 8 hours, followed by a lower temperature (e.g., 60° C.) for a period of 5 days. The mixture is then diluted (e.g., with ethyl acetate) washed, dried, filtered and evaporated to give a crude residue that can be purified conventionally (e.g., using silica gel chromatography) to afford the corresponding compound of Formula I (which, depending on the need to deprotect or further substituted optional substituents may be a precursor to or a product of the invention).

Compounds prepared by the above-described process of the invention can be identified, e.g., by the presence of a detectable amount of Formulae 108, 109, 110, 204, 205, or 304. While it is well known that pharmaceuticals must meet pharmacopoeia standards before approval and/or marketing, and that synthetic reagents, precursors and reaction products (such as 2,3-dihydro-phthalazine-1,4-dione, a reaction product resulting from hydrazine deprotection of a phthalimide-protected compound of Formula I) should not exceed the limits prescribed by pharmacopoeia standards, final compounds prepared by a process of the present invention may have minor, but detectable, amounts of such materials present. For example, the compounds may be in the range of 95% purity with no single impurity greater than 1%. These levels can be detected, e.g., by emission spectroscopy. It is important to monitor the purity of pharmaceutical compounds for the presence of such materials, which presence is additionally disclosed as a method of detecting use of a process of the invention.

A compound of Formula 304 is contacted with an optionally substituted heterocycle of formula 305 to give the corresponding optionally protected compound of Formula I.

An R⁸-protected precursor to Formula I is deprotected.

A compound of Formula 106 or 204 is cyclized.

A racemic mixture of isomers of a compound of Formula I is separated into (R)- and (S)-enantiomers using a chromatography column.

A compound of Formula I is contacted with a pharmaceutically acceptable acid to form the corresponding acid addition salt.

A pharmaceutically acceptable acid addition salt of Formula I is contacted with a base to form the corresponding free base of Formula I.

Representative embodiments of the invention include or employ the compounds of Formula I having the following combinations and permutations of substituent groups (indented/sub-grouped, respectively, in increasing order of particularity). These are presented in support of the appended claims as well as combinations and permutations of substituent groups that may, for the sake of brevity, not be specifically claimed but should be appreciated as encompassed by the teachings of the present disclosure. In that regard, the below-described subsets for each substituent (sometimes referenced by the paragraph's Roman numeral) are intended to apply to that substituent alone or in combination with one, several, or all of the described subsets for the other substituents.

T is optionally substituted lower alkylene or is a covalent bond (i.e., absent).
T is C₁ to C₄ alkylene or C₁ to C₄ alkylene substituted with halo or oxo.
   T is C₁ to C₄ alkylene.
Where T is alkylene having a carbon substituted by a heteroatom, the heteroatom is not bound directly to the bicyclic structure.
   T is aminoalkylene or amidoalkylene.
T is alkylene or alkylene substituted with halo or oxo.

U is optionally substituted lower alkylene or is a covalent bond (i.e., is absent).
U is a covalent bond, C₁ to C₄ alkylene or C₁ to C₄ alkylene substituted with halo or oxo.
   U is a covalent bond or C₁ to C₄ alkylene.
      U is a covalent bond.

W, X, Y and Z are independently chosen from —C═, CH, —N—, —N═, O and S.
Two or three of W, X, Y and Z are —C=or CH.
X, Y or Z is —N═, or W and Z are —N═.
X and Y, taken together with W, Z, and the bridge atoms to which they are bonded, form a nonaromatic, fused, 6-membered heterocyclic ring having a heteroatom chosen from N, O and S.

R¹, R², R³ and R⁴ are each independently chosen from hydrogen, halo, lower alkyl, substituted lower alkyl, lower alkoxy and cyano, or is (are) absent.
R¹, R², R³ and R⁴ are each independently hydrogen, chloro, fluoro, methyl, methoxy, cyano or substituted lower alkyl or absent.
   R¹, R², R³ and R⁴ are each independently hydrogen, chloro, fluoro, methyl, methoxy or cyano or absent.
Where three or four of R¹, R², R³ and R⁴ are hydrogen.
   R¹, R², R³ and R⁴ are each hydrogen or three of R¹, R², R³ and R⁴ are hydrogen and the fourth is halo, methoxy, methyl, cyano or is absent.
      Where halo is chloro.
         Where R³ is hydrogen or chloro.
            Where R³ is chloro.
      Where each R¹, R², R³ and R⁴ is hydrogen or is absent.

R⁵ is optionally substituted aralkyl.
R⁵ is benzyl or substituted benzyl.
   R⁵ is benzyl.

R⁶ is optionally substituted lower alkyl and R^{6′} is hydrogen or optionally substituted lower alkyl.
R^{6′} is hydrogen.
   R⁶ is C₃ to C₅ lower alkyl.
      Lower alkyl is i-propyl, c-propyl or t-butyl.
         Lower alkyl is i-propyl.
            Where the stereogenic center to which R⁶ is attached has an (R)-configuration.
         Where the stereogenic center to which R⁶ is attached has an (R)-configuration.
      Where the stereogenic center to which R⁶ is attached has an (R)-configuration.
   Where the stereogenic center to which R⁶ is attached has an (R)-configuration.
Where the stereogenic center to which R⁶ is attached has an (R)-configuration.
   Where R^{6′} is hydrogen.

R⁷ is substituted alkyl.
Where R⁷ is alkyl substituted with a primary-, secondary- or tertiary-amine.

R⁸ is —C(O)—R⁹ or —C(O)—OR^{9′}.
Where R⁸ is —C(O)—R⁹, in which R⁹ is: optionally substituted aryl (especially phenyl and lower alkyl-, lower alkoxy-, and/or halo-substituted phenyl), optionally substituted aralkyl (especially optionally substituted benzyl and phenylvinyl), aryloxyalkyl (especially phenoxy lower alkyl), optionally substituted heteroaryl, optionally substituted heteroaralkyl, or optionally substituted heteroaryloxyalkyl.
   Where R⁹ is optionally substituted alkyl, optionally substituted aryl or optionally substituted aralkyl.
   Where R⁹ is lower alkoxyalkyl, lower alkyl-substituted phenyl, lower alkoxy-substituted phenyl, halo-substituted phenyl, optionally substituted benzyl, phenoxy lower alkyl, optionally substituted heteroaryl, optionally substituted heteroaralkyl or optionally substituted heteroaryloxyalkyl
      Where R⁹ is lower alkoxyalkyl or substituted phenyl.
         Where R⁹ is methoxy-methyl or p-tolyl.
Where R⁸ is —C(O)—OR^{9′}, in which R^{9′} is optionally substituted aryl (especially phenyl and lower alkyl-, lower alkoxy-, and/or halo-substituted phenyl) or optionally substituted heteroaryl.

Where R⁶ and R⁷ taken together with U and the nitrogen to which R⁷ is bonded form an optionally substituted 5- to 12-membered heterocycle, optionally having up to 2 additional heteroatoms selected from O, N and S.
Where such heterocycle has 5 to 7 ring atoms.
Where T is not a covalent bond, for example, where such heterocycle is optionally substituted 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl.
Where T is a covalent bond provided such heterocycle is not optionally substituted: 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl.

Where R⁷ and R⁸ taken together with the nitrogen to which they are bonded form an optionally substituted 5- to 12-membered heterocycle, optionally having up to 2 additional heteroatoms selected from O, N and S.
Where such heterocycle has 5 to 7 ring atoms.
Where T is not a covalent bond, for example, where such heterocycle is an optionally substituted imidazole or an optionally substituted imidazoline.
   Where R⁷ taken together with R⁸ is 2-(optionally substituted)-4,4-(optionally di-substituted)-4,5-dihydro-imidazol-1-yl.
      Where R⁷ taken together with R⁸ is 2-(optionally substituted aryl)-4,5-dihydro-imidazol-1-yl or 2-(optionally substituted aryl)-4,4-dimethyl-4,5-dihydro-imidazol-1-yl.
         Where R⁷ taken together with R⁸ is 2-(substituted phenyl)-4,5-dihydro-imidazol-1-yl or 2-(substituted phenyl)-4,4-dimethyl-4,5-dihydro-imidazol-1-yl.
            Where R⁷ taken together with R⁸ is 2-(4-methylphenyl)-4,5-dihydro-imidazol-1-yl, 2-(3-fluoro-4-methylphenyl)-4,5-dihydro-imidazol-1-yl, 2-(4-methylphenyl)-4,4-dimethyl-4,5-dihydro-imidazol-1-yl, 2-(3-fluoro-4-methylphenyl)-4,4-dimethyl-4,5-dihydro-imidazol-1-yl.
   Where R⁷ taken together with R⁸ is 2- and/or 4-substituted-imidazol-1-yl.
      Where R⁷ taken together with R⁸ is 2-substituted-imidazol-1-yl.
         Where R⁷ taken together with R⁸ is 2-(optionally substituted phenyl)-imidazol-1-yl.
            Where R⁷ taken together with R⁸ is 2-phenyl-imidazol-1-yl, 2-p-tolyl-imidazol-1-yl, 2-(4-fluoro-phenyl)-imidazol-1-yl, 2-(4-chloro-phenyl)-imidazol-1-yl, or 2-(3-fluoro-4-methyl-phenyl)-imidazol-1-yl.
      Where R⁷ taken together with R⁸ is 2,4-disubstituted-imidazol-1-yl.
         Where R⁷ taken together with R⁸ is 4-(optionally substituted alkyl)-2-(optionally substituted aryl)-imidazol-1-yl.
            Where R⁷ taken together with R⁸ is 4-(Ω-amino-lower alkyl)-2-(optionally substituted phenyl)-imidazol-1-yl, especially: 4-(2-amino-ethyl)-2-phenyl-imidazol-1-yl, 4-(2-amino-ethyl)-2-p-tolyl-imidazol-1-yl, 4-(2-amino-ethyl)-2-(4-fluoro-phenyl)-imidazol-1-yl, 4-(2-amino-ethyl)-2-(4-chloro-phenyl)-imidazol-1-yl, 4-(2-amino-ethyl)-2-(3-fluoro-4-methyl-phenyl)-imidazol-1-yl, 4-(aminomethyl)-2-phenyl-imidazol-1-yl, 4-(aminomethyl)-2-p-tolyl-imidazol-1-yl, 4-(aminomethyl)-2-(4-fluoro-phenyl)-imidazol-1-yl, 4-(aminomethyl)-2-(4-chloro-phenyl)-imidazol-1-yl, or 4-(aminomethyl)-2-(3-fluoro-4-methyl-phenyl)-imidazol-1-yl.

Illustrative of the suitable combinations and permutations of particular substituents are the compounds where T is a optionally substituted lower alkylene, U is a covalent bond, and one or more of W, X, Y, Z, R¹ to R^{9′}, and Rⁱ are as described in paragraphs 91 to 100 above, such as:
Where two or three of W, X, Y and Z are —C═ or CH.
   X, Y or Z is —N═, or W and Z are —N═.
   X and Y, taken together with W, Z and the bridge atoms to which they are bonded, form a nonaromatic, fused, 6-membered heterocyclic ring having a heteroatom chosen from N, O and S.
Where R¹, R², R³ and R⁴ are each hydrogen or three of R¹, R², R³ and R⁴ are hydrogen and the fourth is halo, methoxy, methyl, or cyano or is absent.
   Where R³ is chloro.
   Where R¹, R², R³ and R⁴ are each hydrogen or absent.
Where R^{6′} is hydrogen.
   Where R⁶ is C₃ to C₅ lower alkyl.
      Where R⁶ is i-propyl, c-propyl or t-butyl.
         Where R⁶ is i-propyl.
            Where the stereogenic center to which R⁶ is attached has an (R)-configuration.
         Where the stereogenic center to which R⁶ is attached has an (R)-configuration.
      Where the stereogenic center to which R⁶ is attached has an (R)-configuration.
   Where the stereogenic center to which R⁶ is attached has an (R)-configuration.
Where R⁷ is substituted alkyl.
   Where R⁷ is alkyl substituted with a primary-, secondary- or tertiary-amine.
Where R⁸ is —C(O)—R⁹ or —C(O)—OR^{9′}.
   Where R⁸ is —C(O)—R⁹, in which R⁹ is optionally substituted aryl (such as phenyl and lower alkyl-, lower alkoxy-, and/or halo-substituted phenyl), optionally substituted aralkyl (such as optionally substituted benzyl and phenylvinyl), aryloxyalkyl (such as phenoxy lower alkyl), optionally substituted heteroaryl, optionally substituted heteroaralkyl, or optionally substituted heteroaryloxyalkyl.
   Where R⁸ is —C(O)—OR^{9′}, in which R^{9′} is optionally substituted aryl (such as phenyl and lower alkyl-, lower alkoxy-, and/or halo-substituted phenyl) or optionally substituted heteroaryl.
Where R⁶ and R⁷ taken together with U and the nitrogen to which R⁷ is bonded form an optionally substituted 5- to 12-membered heterocycle, optionally having up to 2 additional heteroatoms selected from O, N and S.
   Where such heterocycle has 5 to 7 ring atoms.
      Where such heterocycle is optionally substituted 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl.
Where R⁷ and R⁸ taken together with the nitrogen to which they are bonded form an optionally substituted 5- to 12-membered heterocycle, optionally having up to 2 additional heteroatoms selected from O, N and S.
   Where such heterocycle has 5 to 7 ring atoms.
      Where such heterocycle is an optionally substituted imidazole or an optionally substituted imidazoline.
Where R⁵ is optionally substituted benzyl.
   R⁵ is benzyl.
      Where two or three of W, X, Y and Z are each —C═ or CH.
         X, Y or Z is —N═, or W and Z are —N═.
         X and Y, taken together with W, Z, and the bridge atoms to which they are bonded, form a nonaromatic, fused, 6-membered heterocyclic ring having a heteroatom chosen from N, O and S.
      Where R¹, R², R³ and R⁴ are each hydrogen or three of R¹, R², R³, and R⁴ are each hydrogen and the fourth is halo, methoxy, methyl, or cyano or absent.
         Where R³ is chloro.
         Where R¹, R², R³ and R⁴ are each hydrogen or absent.
      Where R^{6′} is hydrogen.
         Where R⁶ is C₃ to C₅ lower alkyl.
            Where R⁶ is i-propyl, c-propyl or t-butyl.
            Where R⁶ is i-propyl.
            Where the stereogenic center to which R⁶ is attached has an (R)-configuration.
      Where R⁷ is substituted alkyl.
         Where R⁷ is alkyl substituted with a primary-, secondary- or tertiary-amine.
      Where R⁸ is —C(O)—R⁹ or —C(O)—OR^{9′}.
         Where R⁸ is —C(O)—R⁹, in which R⁹ is: optionally substituted aryl (such as phenyl and lower alkyl-, lower alkoxy-, and/or halo-substituted phenyl), optionally substituted aralkyl (such as optionally substituted benzyl and phenylvinyl), aryloxyalkyl (such as phenoxy lower alkyl), optionally substituted heteroaryl, optionally substituted heteroaralkyl, or optionally substituted heteroaryloxyalkyl.
         Where R⁸ is —C(O)—OR^{9′}, in which R^{9′} is: optionally substituted aryl (such as phenyl and lower alkyl-, lower alkoxy-, and/or halo-substituted phenyl) or optionally substituted heteroaryl.
      Where R⁶ and R⁷ taken together with U and the nitrogen to which R⁷ is bonded form an optionally substituted 5- to 12-membered heterocycle, optionally having up to 2 additional heteroatoms selected from O, N and S.
         Where such heterocycle has 5 to 7 ring atoms.
            Where such heterocycle is optionally substituted 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl.
      Where R⁷ and R⁸ taken together with the nitrogen to which they are bonded form an optionally substituted 5- to 12-membered heterocycle, optionally having up to 2 additional heteroatoms selected from O, N and S.
         Where such heterocycle has 5 to 7 ring atoms.
            Where such heterocycle is an optionally substituted imidazole or an optionally substituted imidazoline.
   Where two or three of W, X, Y and Z are each —C=or CH.
      X, Y or Z is —N═, or W and Z are —N═.
      X and Y, taken together with W, Z and the bridge atoms to which they are bonded, form a nonaromatic, fused, 6-membered heterocyclic ring having a heteroatom chosen from N, O and S.
   Where R¹, R², R³ and R⁴ are hydrogen or three of R¹, R², R³ and R⁴ are hydrogen and the fourth is halo, methoxy, methyl, or cyano.
      Where R³ is chloro.
      Where R¹, R², R³ and R⁴ are hydrogen.
   Where R^{6′} is hydrogen.
      Where R⁶ is C₃ to C₅ lower alkyl.
         Where R⁶ is i-propyl, c-propyl or t-butyl.
            Where R⁶ is i-propyl.
            Where the stereogenic center to which R⁶ is attached has an (R)-configuration.
   Where R⁷ is substituted alkyl.
      Where R⁷ is alkyl substituted with a primary-, secondary- or tertiary-amine.
   Where R⁸ is —C(O)—R⁹ or —C(O)—OR^{9′}.
      Where R⁸ is —C(O)—R⁹, in which R⁹ is: optionally substituted aryl (such as phenyl and lower alkyl-, lower alkoxy-, and/or halo-substituted phenyl), optionally substituted aralkyl (such as optionally substituted benzyl and phenylvinyl), aryloxyalkyl (such as phenoxy lower alkyl), optionally substituted heteroaryl, optionally substituted heteroaralkyl, or optionally substituted heteroaryloxyalkyl.
      Where R⁸ is —C(O)—OR⁹, in which R^{9′} is: optionally substituted aryl (such as phenyl and lower alkyl-, lower alkoxy-, and/or halo-substituted phenyl) or optionally substituted heteroaryl.
   Where R⁶ and R⁷ taken together with U and the nitrogen to which R⁷ is bonded form an optionally substituted 5- to 12-membered heterocycle, optionally having up to 2 additional heteroatoms selected from O, N and S.
      Where such heterocycle has 5 to 7 ring atoms.
         Where such heterocycle is optionally substituted 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl.
   Where R⁷ and R⁸ taken together with the nitrogen to which they are bonded form an optionally substituted 5- to 12-membered heterocycle, optionally having up to 2 additional heteroatoms selected from O, N and S.
      Where such heterocycle has 5 to 7 ring atoms.
         Where such heterocycle is an optionally substituted imidazole or an optionally substituted imidazoline.
            Thus, the compounds where T is optionally substituted lower alkylene and U is a covalent bond, including those where the above-described groupings and sub-groups of substituents are taken individually and/or combined together as illustrated with regard to those compounds where R⁵ is optionally substituted benzyl, are particularly suitable for practice of the present invention.

Some embodiments are drawn to at least one chemical entity chosen from a compound of Formula I and pharmaceutically acceptable salts, solvates, crystal forms, diastereomers, and prodrugs thereof chosen from:
N-(3-amino-propyl)-N-[1-(3-benzyl-4-oxo-3,4-dihydro-pyrido[2,3-d]pyrimidin-2-ylethyl-2-methyl-propyl]-4-methyl-benzamide,
N-(3-amino-propyl)-N-[1-(3-benzyl-4-oxo-3,4-dihydro-pyrido[4,3-d]pyrimidin-2-ylethyl)-2-methyl-propyl]-4-methyl-benzamide,
N-(3-amino-propyl)-N-[1-(3-benzyl-4-oxo-3,4-dihydro-pyrido[3,4-d]pyrimidin-2-ylmethyl)-2-methyl-propyl]-4-methyl-benzamide,
N-(3-amino-propyl)-N-[1-(3-benzyl-4-oxo-3,4-dihydro-pyrido[2,3-d]pyrimidin-2-ylmethyl)-2-methyl-propyl]-4-methyl-benzamide,
2-{2-[6-(2-amino-ethyl)-3-methyl-1-(4-methyl-benzoyl)-piperidin-2-yl]-ethyl}-3-benzyl-5,6-dihydro-3H-pyrido[3,4-d]pyrimidin-4-one,
N-(3-amino-propyl)-N-{2-[(3-benzyl-4-oxo-3,4,5,7-tetrahydro-furo[3,4-d]pyrimidin-2-ylmethyl)-methyl-amino]-propyl}-4-methyl-benzenesulfonamide,
N-(3-amino-propyl)-N-[1-(3-benzyl-4-oxo-3,4-dihydro-pyrido[3,2-d]pyrimidin-2-ylpropyl)-2-methyl-propyl]-4-methyl-benzamide, and
N-(3-amino-propyl)-N-[1-(3-benzyl-4-oxo-3,4-dihydro-pteridin-2-ylmethyl)-2-methyl-propyl]-4-methyl-benzamide.

The compounds of the invention find use in a variety of applications, including as therapeutic active agents, in the practice of the methods of treatment, in compositions, such as pharmaceutical formulations and in methods for the manufacture of pharmaceutical formulations, and as intermediates in the synthesis of such therapeutic active agents.

As will be appreciated by those in the art, mitosis can be altered in a variety of ways, that is, one can affect mitosis either by increasing, decreasing or otherwise interfering with the activity of a component in the mitotic pathway. Stated differently, mitosis can be affected (e.g., disrupted) by disturbing equilibrium, either by inhibiting or activating certain mitotic components. Similar approaches can be used to alter meiosis.

In one embodiment of the invention, the compounds of Formula I can be used to inhibit mitotic spindle formation. Such inhibition may take the form of lessening a mitotic kinesin's organization of microtubules into bipolar structures, increasing or decreasing spindle pole separation, and/or inducing mitotic spindle dysfunction. The compounds of Formula I are useful to bind to and/or inhibit the activity of a mitotic kinesin, KSP, such as human KSP, although KSP kinesins from other organisms may also be used. Also included within the definition of the term “KS P” for these purposes are variants and/or fragments of KSP. See, U.S. Pat. No. 6,437,115. While other mitotic kinesins may be used in the present invention, the compounds of the invention have been shown to have specificity for KSP. Contacting a compound of the invention with a KSP kinesin, such as human KSP kinesin, can lead to diminished KSP-mediated ATP hydrolysis activity and/or diminished KSP-mediated mitotic spindle formation activity. Meiotic spindles can be similarly disrupted.

In another embodiment, the compounds Formula I can be used to modulate one or more other human mitotic kinesins, in addition to KSP, including HSET (see, U.S. Pat. No. 6,361,993); MCAK (see, U.S. Pat. No. 6,331,424); CENP-E (see, U.S. Pat. No. 6,645,748); Kif4 (see, U.S. Pat. No. 6,440,684); MKLP1 (see, U.S. Pat. No. 6,448,025); Kif15 (see, U.S. Pat. No. 6,355,466); Kid (see, U.S. Pat. No. 6,387,644); Mpp1, CMKrp, KinI-3 (see, U.S. Pat. No. 6,461,855); Kip3a (see, U.S. Pat. No. 6,680,369); Kip3d (see, U.S. Pat. No. 6,492,151); and RabK6.

Therapeutic uses facilitated by the mitotic kinesin-inhibitory activity of the compounds of the present invention include the treatment of disorders associated with cell proliferation. Disease states that can be treated by the methods, pharmaceutical formulations, and compounds provided herein include, but are not limited to, cancer (further discussed below), autoimmune disease, arthritis, graft rejection, inflammatory bowel disease, proliferation induced after medical procedures, including, but not limited to, surgery, angioplasty, and the like. In one embodiment, the invention includes application to cells or individuals afflicted or impending afflication with any one of these disorders or states.

The compounds, pharmaceutical formulations and methods provided herein are useful for the treatment of cancer including solid tumors such as skin, breast, brain, cervical carcinomas, testicular carcinomas, etc. Cancers that can be treated include, but are not limited to:
Cardiac: sarcoma (angiosarcoma, fibrosarcoma, rhabdomyosarcoma, liposarcoma), myxoma, rhabdomyoma, fibroma, lipoma and teratoma;
Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;
Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);
Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);
Liver: hepatoma (hepatocellular carcinoma), cholangiocarcinoma, hepatoblastoma, angiosarcoma, hepatocellular adenoma, hemangioma;
Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;
Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma);
Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma], fallopian tubes (carcinoma);
Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma];
Skin: malignant melanoma, basal cell carcinoma, squamous cell carcinoma, Karposi's sarcoma, moles dysplastic nevi, lipoma, angioma, dermatofibroma, keloids, psoriasis; and
Adrenal glands: neuroblastoma.
   As used herein, treatment of cancer includes treatment of cancerous cells, including cells afflicted by any one of the above-identified conditions.

Another useful aspect of the invention is a kit having at least one chemical entity chosen from a compound of Formula I and pharmaceutically acceptable salts, solvates, crystal forms, diastereomers, and prodrugs thereof, and a package insert or other labeling including directions treating a cellular proliferative disease by administering an effective amount of the chemical entity. The chemical entity in the kits of the invention may be provided as one or more doses for a course of treatment for a cellular proliferative disease, each dose being a pharmaceutical formulation including a pharmaceutically accepted excipient and at least one chemical entity chosen from a compound of Formula I and pharmaceutically acceptable salts, solvates, crystal forms, diastereomers, and prodrugs thereof.

To assay activity, generally, either KSP or a compound of Formula I is non-diffusably bound to an insoluble support having isolated sample receiving areas. The insoluble support can be made of any material to which the compounds can be bound, is readily separated from soluble material, and is otherwise compatible with the overall method of screening. The surface of such supports can be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are convenient because a large number of assays can be carried out simultaneously, using small amounts of reagents and samples. The particular manner of binding of the compound is not crucial so long as it is compatible with the reagents and overall methods of the invention, maintains the activity of the compound and is nondiffusable. Methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas can then be blocked through incubation with bovine serum albumin (BSA), casein or other innocuous protein or other moiety.

The compounds of Formula I can be used on their own to modulate the activity of a mitotic kinesin, such as KSP. In this embodiment, a compound of Formula I is combined with KSP and the activity of KSP is assayed. Measurable kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes, such as kinases or proteases; and specific kinesin cellular activities such as spindle pole separation.

Methods of performing motility assays are well known to those of skill in the art. See, e.g., Hall, et al. (1996), Biophys. J., 71: 3467-3476, Turner et al., 1996, Anal. Biochem. 242 (1): 20-5; Gittes et al., 1996, Biophys. J. 70(1): 418-29; Shirakawa et al., 1995, J. Exp. Biol. 198: 1809-15; Winkelmann et al., 1995, Biophys. J. 68: 2444-53; Winkelmann et al., 1995, Biophys. J. 68: 72S.

Methods known in the art for determining ATPase hydrolysis activity also can be used. Solution based assays are suitable (see, U.S. Pat. No. 6,410,254); alternatively, conventional methods are used. For example, Pᵢ release from kinesin can be quantified. In one embodiment, the ATPase hydrolysis activity assay utilizes 0.3 M PCA (perchloric acid) and malachite green reagent (8.27 mM sodium molybdate II, 0.33 mM malachite green oxalate, and 0.8 mM Triton X-100). To perform the assay, 10 μL of reaction is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. When phosphate standards are used, absorbance readings can be converted to mM Pᵢ and plotted over time. Other ATPase assays known in the art include the luciferase assay.

ATPase activity of kinesin motor domains also can be used to monitor the effects of modulating agents. In one embodiment, ATPase assays of kinesin are performed in the absence of microtubules. In another embodiment, the ATPase assays are performed in the presence of microtubules. Different types of modulating agents can be detected in the above assays. In one embodiment, the effect of a modulating agent is independent of the concentration of microtubules and ATP. In another embodiment, the effect of the agents on kinesin ATPase can be decreased by increasing the concentrations of ATP, microtubules, or both. In yet another embodiment, the effect of the modulating agent is increased by increasing concentrations of ATP, microtubules, or both.

Agents that modulate the biochemical activity of KSP in vitro may then be screened in vivo. Methods for testing such agents in vivo include assays of cell cycle distribution, cell viability, or the presence, morphology, activity, distribution or amount of mitotic spindles. Methods for monitoring cell cycle distribution of a cell population, for example, by flow cytometry, are well known to those skilled in the art, as are methods for determining cell viability. See, for example, WO 01/31335, entitled “Methods of Screening for Modulators of Cell Proliferation and Methods of Diagnosing Cell Proliferation States.”

In addition to the assays described above, microscopic methods for monitoring spindle formation and malformation are well known to those of skill in the art (see, e.g., Whitehead and Rattner (1998), J. Cell Sci. 111: 2551-61; Galgio et al, (1996) J. Cell Biol., 135: 399-414).

The compounds of Formula I inhibit KSP kinesin. One measure of inhibition, IC₅₀, is defined as the concentration of the compound at which the activity of KSP is decreased by fifty percent. Suitable compounds have IC₅₀'s of less than about 1 mM, with more suitable compounds having IC₅₀'s of less than about 100 μM. IC₅₀'s of less than about 10 nM can be attained by certain compounds of the invention. It will be appreciated that a smaller IC₅₀ is generally considered advantageous. Measurement of IC₅₀ may be done using an ATPase assay.

Another measure of inhibition is Kᵢ. For compounds with IC₅₀'s less than 1 μM, the Kᵢ or K_{d} is defined as the dissociation rate constant for the interaction of the test compound with KSP. Suitable compounds have Kᵢ's of less than about 100 μM, for example, less than about 10 μM. Kᵢ's of less than about 10 nM can be attained by certain compounds of Formula I. It will be appreciated that a smaller Kᵢ is generally considered advantageous. The Kᵢ for a compound is determined from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ 1 - \frac{( {E}_{0} + {I}_{0} + {K}_{d} ) - \sqrt{{( {E}_{0} + {I}_{0} + {K}_{d} )}^{2} - 4 {E}_{0} {I}_{0}}}{2 {E}_{0}} ]$$

Where V is the observed rate, Vₘₐₓ is the rate of the free enzyme, 10 is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Anti-proliferative compounds that have been successfully applied in the clinic to treatment of cancer (cancer chemotherapeutics) have GI₅₀'s that vary greatly. For example, in A549 cells, the GI₅₀ of paclitaxel is 4 nM, the GI₅₀ of doxorubicin is 63 nM, the GI₅₀ of 5-fluorouracil is 1 μM, and the GI₅₀ of hydroxyurea is 500 μM (data provided by National Cancer Institute, Developmental Therapeutic Program, available at http://dtp.nci.nih.gov/). Therefore, compounds that inhibit cellular proliferation at virtually any concentration may be useful. Suitable compounds have GI₅₀'s of less than about 1 mM and more suitable compounds have a GI₅₀ of less than about 10 μM. GI₅₀'s of less than about 10 nM can be attained by certain compounds of Formula I, it being appreciated that a smaller GI₅₀ is generally considered advantageous. Measurement of GI₅₀ is done using a cell proliferation assay.

Testing for growth inhibition using cell lines (such as MCF-7/ADR-RES and HCT1 5) that express P-glycoprotein (also known as Multi-drug Resistance, or MDR⁺), which conveys resistance to other chemotherapeutic drugs, such as pacilitaxel, can identify anti-mitotic agents that inhibit cell proliferation and are not subject to resistance by overexpression of MDR⁺ by drug-resistant tumor lines.

In vitro potency of small molecule inhibitors can be determined by assaying human ovarian cancer cells (SKOV3) for viability following a 72-hour exposure to a 9-point dilution series of compound. Cell viability is determined by measuring the absorbance of formazon, a product formed by the bioreduction of MTS/PMS, a commercially available reagent. Each point on the dose-response curve is calculated as a percent of untreated control cells at 72 hours minus background absorption (complete cell kill).

To employ the compounds of Formula I in a method of screening for compounds that bind to KSP kinesin, the KSP may be bound to a support, and a compound of Formula I or composition comprising a compound of Formula I is added to the assay. Alternatively, a composition comprising a compound of Formula I bound to a solid support can be made, and KSP added to the assay. Classes of compounds among which novel binding agents may be sought include specific antibodies, non-natural binding agents identified in screens of chemical libraries, peptide analogs, etc. Screening assays for candidate agents that have a low toxicity for human cells may be employed. A wide variety of assays may be used for this purpose, including labeled in vitro protein-protein binding assays, electrophoretic mobility shift assays, immunoassays for protein binding, functional assays (phosphorylation assays, etc.), and the like.

The determination of the binding of the mitotic agent to KSP may be done in a number of ways. In one embodiment, a compound Formula I is labeled, for example, with a fluorescent or radioactive moiety and binding determined directly. For example, this may be done by attaching all or a portion of KSP to a solid support, adding a labeled compound (for example a compound of the invention in which at least one atom has been replaced by a detectable isotope), washing off excess reagent, and determining whether the amount of the label is that present on the solid support. Various blocking and washing steps may be utilized as is known in the art.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin, etc. For the specific binding members, the complementary member would typically be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the anti-mitotic agents.

The compounds of Formula I may also be used as competitors to screen for additional drug candidates. The terms “candidate agent,” “drug candidate,” and grammatical equivalents, as used herein, describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, particular embodiments exclude molecules already known to bind to that protein, for example, polymer structures such as microtubules, and energy sources such as ATP. Certain embodiments of assays herein include candidate agents that do not bind the cellular proliferation protein in its endogenous native state termed herein as “exogenous” agents. In another embodiment, exogenous agents further exclude antibodies to KSP.

Candidate agents can encompass numerous chemical classes, though typically they are organic molecules, such as small organic compounds having a molecular weight of more than 100 and less than about 2,500 daltons. Candidate agents comprise functional groups necessary for structural interaction with proteins, such as hydrogen bonding and lipophilic binding, and typically include at least an amine, carbonyl, hydroxyl, ether, or carboxyl group, especially at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous methods are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, and amidification to produce structural analogs.

Competitive screening assays can be performed done by combining KSP and a drug candidate in a first sample. A second sample may be made combining a compound of Formula I, KSP and a drug candidate. This may be performed in either the presence or absence of microtubules. The binding of the drug candidate is determined for both samples, and a change or difference in binding between the two samples indicates the presence of an agent capable of binding to KSP and potentially modulating its activity. That is, if the binding of the drug candidate is different in the second sample relative to the first sample, the drug candidate is capable of binding to KSP.

In one embodiment, the binding of the candidate agent is determined through the use of competitive binding assays. In this embodiment, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In one embodiment, the candidate agent is labeled. Either the candidate agent, or the competitor, or both, is added first to KSP for a time sufficient to allow binding, if present. Incubations can be performed at any temperature that facilitates optimal activity, typically between 4 and 40° C. Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to determine binding.

In one embodiment, the competitor is added first, followed by the candidate agent. Displacement of the competitor is an indication the candidate agent is binding to KSP and thus is capable of binding to, and potentially modulating, the activity of KSP. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

It may be of value to identify the binding site of KSP. This can be done in a variety of ways. In one embodiment, after a compound has been identified as binding to KSP, the KSP may be fragmented or modified and the assays repeated to identify the necessary components for binding.

Modulation is tested by screening for candidate agents capable of modulating the activity of KSP comprising the steps of combining a candidate agent with KSP, as above, and determining an alteration in the biological activity of KSP. Thus, in this embodiment, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to native KSP, but cannot bind to modified KSP.

Positive controls and negative controls may be used in the assays. All control and test samples may be performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted with a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

The chemical entity chosen from a compound of Formula I and pharmaceutically acceptable salts, solvates, crystal forms, diastereomers, and prodrugs thereof is administered at a therapeutically effective dosage, e.g., a dosage sufficient to provide treatment for the disease states previously described. Human dosage levels are typically determined by escalating dose ranging studies conducted in accordance with current Good Clinical Practice, FDA and local guidelines. The amount of active compound administered will, of course, be dependent on the subject and disease state being treated, the severity of the affliction, the manner and schedule of administration and the judgment of the prescribing physician.

The administration of the compounds and pharmaceutical formulations of the present invention can be done in a variety of ways, including, but not limited to, orally, subcutaneously, intravenously, intranasally, transdermally, intraperitoneally, intramuscularly, intrapulmonary, vaginally, rectally, or intraocularly. In some instances, for example, in the treatment of wounds and inflammation, the compound or composition may be directly applied as a solution or spray.

Pharmaceutical formulations include at least one chemical entity chosen from a compound of Formula I and pharmaceutically acceptable salts, solvates, crystal forms, diastereomers, and prodrugs thereof, and one or more pharmaceutically acceptable excipients. As is known in the art, pharmaceutical excipients are secondary ingredients that function to enable or enhance the delivery of a drug or medicine in a variety of dosage forms (e.g.: oral forms such as tablets, capsules, and liquids; topical forms such as dermal, opthalmic, and otic forms; suppositories; injectables; respiratory forms and the like). Pharmaceutical excipients include inert or inactive ingredients, synergists or chemicals that substantively contribute to the medicinal effects of the active ingredient. For example, pharmaceutical excipients may function to improve flow characteristics, product uniformity, stability, taste, or appearance, to ease handling and administration of dose, for convenience of use, or to control bioavailability. While pharmaceutical excipients are commonly described as being inert or inactive, it is appreciated in the art that there is a relationship between the properties of the pharmaceutical excipients and the dosage forms containing them.

Pharmaceutical excipients suitable for use as carriers or diluents are well known in the art, and may be used in a variety of formulations. See, e.g., Remington's Pharmaceutical Sciences, 18th Edition, A. R. Gennaro, Editor, Mack Publishing Company (1990); Remington: The Science and Practice of Pharmacy, 20th Edition, A. R. Gennaro, Editor, Lippincott Williams & Wilkins (2000); Handbook of Pharmaceutical Excipients, 3rd Edition, A. H. Kibbe, Editor, American Pharmaceutical Association, and Pharmaceutical Press (2000); and Handbook of Pharmaceutical Additives, compiled by Michael and Irene Ash, Gower (1995). The concentration of a therapeutically active agent in a formulation can vary widely, from about 0.1 to 99.9 wt. %, depending on the nature of the formulation.

Oral solid dosage forms such as tablets will typically comprise one or more pharmaceutical excipients, which may for example help impart satisfactory processing and compression characteristics, or provide additional desirable physical characteristics to the tablet. Such pharmaceutical excipients may be selected from diluents, binders, glidants, lubricants, disintegrants, colorants, flavorants, sweetening agents, polymers, waxes, and other solubility-modulating materials.

Dosage forms for parenteral administration will generally comprise fluids, such as intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy, and include:
alcohol, e.g., 5% alcohol (e.g., in dextrose and water (“D/W”) or D/W in normal saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., ⅙ M; and
sterile water for injection
   The pH of such IV fluids may vary, and will typically be from 3.5 to 8 as known in the art.

The at least one chemical entity chosen from a compound of Formula I and pharmaceutically acceptable salts, solvates, crystal forms, diastereomers, and prodrugs thereof, can be administered alone or in combination with other treatments, i.e., radiation, or other therapeutic agents, such as the taxane class of agents that appear to act on microtubule formation or the camptothecin class of topoisomerase I inhibitors. When so-used, other therapeutic agents can be administered before, concurrently (whether in separate dosage forms or in a combined dosage form), or after administration of an active agent of the present invention.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes.

### EXAMPLES

### Example 1

### Induction of Mitotic Arrest in Cell Populations Treated with a KSP Inhibitor

Flow cytometry analysis to determine cell cycle stage by measuring DNA content is performed as follows. Skov-3 cells (human ovarian cancer) are split 1:10 for plating in 10 cm dishes and grown to subconfluence with RPMI 1640 medium containing 5% fetal bovine serum (FBS). The cells are then treated with either 10 nM paclitaxel, 400 nM test compound, 200 nM test compound, or 0.25% DMSO (vehicle for compounds) for 24 hours. A well known anti-mitotic agent, such as paclitaxel, is used as a positive control. Cells are then rinsed off the plates with phospate buffered saline (PBS) containing 5 mM EDTA, pelleted, washed once in PBS containing 1% FCS, and then fixed overnight in 85% ethanol at 4° C. Before analysis, the cells are pelleted, washed once, and stained in a solution of 10 μg propidium iodide and 250 μg of ribonuclease A (RNAse A) per milliliter at 37° C. for half an hour. Flow cytometry analysis is performed on a Becton-Dickinson FACScan, and data from 10,000 cells per sample is analyzed with Modfit software.

Monopolar Spindle Formation Following Application of a Quinazolinone KSP Inhibitor

To determine the nature of G2/M accumulation, human tumor cell lines Skov-3 (ovarian), HeLa (cervical), and A549 (lung) are plated in 96-well plates at densities of 4,000 cells per well (SKOV-3 & HeLa) or 8,000 cells per well (A549), allowed to adhere for 24 hours, and treated with various concentrations of the test compounds for 24 hours. Cells are fixed in 4% formaldehyde and stained with anti-tubulin antibodies (subsequently recognized using fluorescently-labeled secondary antibody) and Hoechst dye (which stains DNA). The cells can be visually inspected to assess the effects of the test compounds. For example, microinjection of anti-KSP antibodies causes mitotic arrest with arrested cells displaying monopolar spindles.

### Example 2

### Inhibition of Cellular Proliferation in Tumor Cell Lines Treated with KSP Inhibitors

Cells are plated in 96-well plates at densities from 1000-2500 cells/well (depending on the cell line) and allowed to adhere/grow for 24 hours. The cells are then treated with various concentrations of test compound for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450; see Promega product catalog #G3580, CellTiter 96® AQᵤₑₒᵤₛ One Solution Cell Proliferation Assay) is used to determine the number of viable cells at T₀ and the number of cells remaining after 48 hours compound exposure. The number of cells remaining after 48 hours is compared to the number of viable cells at the time of test compound addition, allowing for calculation of growth inhibition. The growth over 48 hours of cells in control wells treated with vehicle only (0.25% DMSO) is considered 100% growth and the growth of cells in wells with compounds is compared to this. Active KSP inhibitors inhibit cell proliferation in one or more human tumor cell lines of the following tumor types: lung (NCI-H460, A549), breast (MDA-MB-231, MCF-7, MCF-7/ADR-RES), colon (HT29, HCT15), ovarian (SKOV-3, OVCAR-3), leukemia (HL-60(TB), K-562), central nervous system (SF-268), renal (A498), osteosarcoma (U2-OS), cervical (HeLa), and mouse tumor line (B16, melanoma).

Calculation Of GI₅₀: A GI₅₀ is calculated by plotting the concentration of compound in vs the percentage of cell growth of cell growth in treated wells. The GI₅₀ calculated for the compounds is the estimated concentration at which growth is inhibited by 50% compared to control, i.e., the concentration at which:
100×[(Treated₄₈−T₀)/(Control₄₈−T₀)]=50.
All concentrations of compounds are tested in duplicate and controls are averaged over 12 wells. A very similar 96-well plate layout and GI₅₀ calculation scheme is used by the National Cancer Institute (see Monks, et al., J. Natl. Cancer Inst. 83: 757-766 (1991)). However, the method by which the National Cancer Institute quantitates cell number using a method that does not use MTS.

Calculation Of IC₅₀: The compound's IC₅₀ for KSP activity is measured using an ATPase assay. The following solutions are used: Solution 1 consists of 3 mM phosphoenolpyruvate potassium salt (Sigma P-7127), 2 mM ATP (Sigma A-3377), 1 mM IDTT (Sigma D-9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT400301), and 1 mM EGTA (Sigma E3889). Solution 2 consists of 1 mM NADH (Sigma N8129), 0.2 mg/ml BSA (Sigma A7906), pyruvate kinase 7 U/ml, L-lactate dehydrogenase 10 U/ml (Sigma PO₂₉₄), 100 nM KSP motor domain, 50 μg/ml microtubules, 1 mM DTT (Sigma D9779), 5 μM paclitaxel (Sigma T-7402), 10 ppm antifoam 289 (Sigma A-8436), 25 mM Pipes/KOH pH 6.8 (Sigma P6757), 2 mM MgCl2 (VWR JT4003-01), and 1 mM EGTA (Sigma E3889). Serial dilutions (8-12 two-fold dilutions) of the composition are made in a 96-well microtiter plate (Corning Costar 3695) using Solution 1. Following serial dilution each well has 50 Ill of Solution 1. The reaction is started by adding 501 μl of Solution 2 to each well. This can be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x is the compound concentration.

### Example 3

### Inhibition of Cellular Viability in Tumor Cell Lines Treated with KSP Inhibitors

Materials and Solutions:
Cells: SKOV3, Ovarian Cancer (human).
Media: Phenol Red Free RPMI+5% Fetal Bovine Serum+2 mM L-glutamine.
Colorimetric Agent for Determining Cell Viability: Promega MTS tetrazolium compound.
Control Compound for max cell kill: Topotecan, 1 μM.

Procedure: Day 1—Cell Plating: Adherent SKOV3 cells are washed with 10 mL of PBS followed by the addition of 2 mL of 0.25% trypsin and incubation for 5 minutes at 37° C. The cells are rinsed from the flask using 8 mL of media (phenol red-free RPMI+5% FBS) and transferred to fresh flask. Cell concentration is determined using a Coulter counter and the appropriate volume of cells to achieve 1000 cells/100 μL is calculated. 100 μL of media cell suspension (adjusted to 1000 cells/100 μL) is added to all wells of 96-well plates, followed by incubation for 18 to 24 hours at 37° C., 100% humidity, and 5% CO₂, allowing the cells to adhere to the plates.

Procedure: Day 2—Compound Addition: To one column of the wells of an autoclaved assay block are added an initial 2.5 μL of test compound(s) at 400× the highest desired concentration. 1.25 μL of 400× (400 μM) topotecan is added to other wells (ODs from these wells are used to subtract out for background absorbance of dead cells and vehicle). 500 μL of media without DMSO are added to the wells containing test compound, and 250 μL to the topotecan wells. 250 μL of media+0.5% DMSO is added to all remaining wells, into which the test compound(s) are serially diluted. By row, compound-containing media is replica plated (in duplicate) from the assay block to the corresponding cell plates. The cell plates are incubated for 72 hours at 37° C., 100% humidity, and 5% CO₂.

Procedure: Day 4—MTS Addition and OD Reading: The plates are removed from the incubator and 40 μl MTS/PMS is added to each well. Plates are then incubated for 120 minutes at 37° C., 100% humidity, 5% CO₂, followed by reading the ODs at 490 nm after a 5 second shaking cycle in a 96-well spectrophotometer.

Data Analysis The normalized % of control (absorbance−background) is calculated and an XLfit is used to generate a dose-response curve from which the concentration of compound required to inhibit viability by 50% is determined.

The compounds of Formula I show activity when tested in one or more of the methods described in Examples 1, 2 and 3.

While the present invention has been described with reference to specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto. All patents and publications cited above are hereby incorporated by reference.

1. At least one chemical entity chosen from a compound of Formula I: 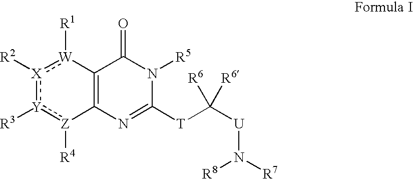
and pharmaceutically acceptable salts, solvates, crystal forms, diastereomers, and prodrugs thereof, wherein
T and U are independently absent or optionally substituted lower alkylene;
W, X and Y are independently chosen from —N═, —N—, —C═, CH, CRⁱ, O and S;
Z is chosen from —N═, —N—, —C═, CH, CRⁱ, O and S, or is absent, provided that:
no more than two of W, X, Y and Z are both —N═, O or S, and
W, X, Y or Z can be O or S only when the ring they form is not aromatic, or W, X or Y can be O or S when Z is absent;
Rⁱ is chosen from optionally substituted alkyl, optionally substituted alkoxy, optionally substituted aryl, optionally substituted heteroaryl, halo and cyano;
R¹, R², R³ and R⁴ are independently chosen from hydrogen, hydroxy, optionally substituted alkyl, optionally substituted alkoxy, alkylsulfonyl, alkylsulfonamido, carboxamido, alkylthio, aminocarbonyl, optionally substituted aryl, optionally substituted heteroaryl, halo, nitro and cyano, provided that R¹, R² or R³ is absent when W, X or Y, respectively, is —N═, S, or O, and R⁴ is absent when Z is —N═, S, or O or is absent;
R⁵ is chosen from optionally substituted alkyl, optionally substituted aryl and optionally substituted heteroaryl;
R⁶ is chosen from optionally substituted alkyl and optionally substituted aryl,
R^{6′} is chosen from hydrogen, optionally substituted alkyl and optionally substituted aryl;
R⁷ is chosen from optionally substituted alkyl, optionally substituted aryl and optionally substituted heteroaryl; and
R⁸ is chosen from hydrogen, —C(O)—R⁹, —S(O)₂—R^{9′}, —CH₂—R⁹, —C(O)—O—R^{9′}, —C(O)—NH—R⁹ or —S(O)₂—NH—R⁹, wherein:
R⁹ is chosen from hydrogen, optionally substituted alkyl, optionally substituted aryl and optionally substituted heteroaryl, and
R^{9′} is chosen from optionally substituted alkyl, optionally substituted aryl and optionally substituted heteroaryl;
or alternatively:
R⁶ and R^{6′}, taken together with the carbon to which they are bonded, form an optionally substituted cycloalkyl or optionally substituted heterocycloalkyl having 5 to 7 ring atoms; or
R⁶ and R⁷ taken together with the nitrogen to which R⁷ is bonded form an optionally substituted 5- to 12-membered heterocycle having up to 2 additional heteroatoms selected from O, N and S, provided that such heterocycle is not optionally substituted: 5-oxo-pyrrolidin-2-yl, 6-oxo-piperidin-2-yl, 2-oxo-hexahydro-pyrimidin-4-yl, pyrrolidin-2-yl, pyrrolidin-3-yl, piperidin-2-yl, piperidin-3-yl or piperidin-4-yl; or
R⁷ and R⁸, taken together with the nitrogen to which they are bonded form an optionally substituted 5- to 12-membered heterocycle having up to 2 additional heteroatoms selected from O, N and S, provided that such heterocycle is not optionally substituted: 1-piperazin-1-yl, 1-[1,4]diazepan-1-yl, 2-oxo-[1,4]diazepan-1-yl, 7-oxo-[1,4]diazepan-1-yl, imidazol-1-yl or imidazolin-1-yl; or
R⁶ taken together with R⁷, and R^{6′} taken together with R⁸ and the nitrogen to which R⁷ and R⁸ are bonded form an optionally substituted 10- to 13-membered fused heterocycle having up to 2 additional heteroatoms selected from O, N and S,
provided that:
at least one of W, X, Y or Z is other than —C═; and
when T is a covalent bond, then Z is present and at least one of W, X, Y or Z is O or S; and
when T is a covalent bond and R⁸ is hydrogen, —C(O)—R⁹, —S(O)₂—R^{9′}, —CH₂—R⁹, —C(O)—O—R^{9′}, —C(O)—NH—R⁹ or —S(O)₂—NH—R⁹, and when R⁷ and R⁸ are optionally substituted imidazolyl or optionally substituted imidazolinyl, then at least one of W, X, Y or Z is —N—, CH, CRⁱ, O or S.

2. At least one chemical entity of claim 1 chosen from a compound of Formula I: 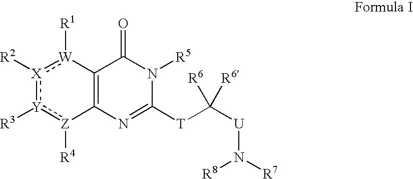
and pharmaceutically acceptable salts, solvates, crystal forms, diastereomers, and prodrugs thereof, wherein
T is optionally substituted lower alkylene;
U is chosen from a covalent bond or optionally substituted lower alkylene;
W, X and Y are independently chosen from —N═, N, —C═, CH, CRⁱ, O and S;
Z is chosen from —N═, N, —C═, CH, CRⁱ, O and S, or is absent, provided that:
no more than two of W, X, Y and Z are both —N═, O or S, and
W, X, Y or Z can be O or S only when the ring they form is not aromatic, or W, X or Y can be O or S when Z is absent;
Rⁱ is chosen from optionally substituted alkyl, optionally substituted alkoxy, optionally substituted aryl, optionally substituted heteroaryl, halogen and cyano;
R¹, R² R³ and R⁴ are independently chosen from hydrogen, hydroxy, optionally substituted alkyl, optionally substituted alkoxy, alkylsulfonyl, alkylsulfonamido, carboxamido, alkylthio, aminocarbonyl, optionally substituted aryl, optionally substituted heteroaryl, halogen, nitro and cyano, provided that R¹, R² or R³ is absent where W, X or Y, respectively, is —N═, S, or O, and R⁴ is absent where Z is —N═, S, O or is absent;
R⁵ is chosen from optionally substituted alkyl, optionally substituted aryl and optionally substituted heteroaryl;
R⁶ is chosen from optionally substituted alkyl and optionally substituted aryl,
R^{6′} is chosen from hydrogen, optionally substituted alkyl and optionally substituted aryl;
R⁷ is chosen from optionally substituted alkyl, optionally substituted aryl and optionally substituted heteroaryl; and
R⁸ is chosen from hydrogen, —C(O)—R⁹, —S(O)₂—R^{9′}, —CH₂—R⁹, —C(O)—O—R^{9′}, —C(O)—NH—R⁹ or —S(O)₂—NH—R⁹, in which:
R⁹ is chosen from hydrogen, optionally substituted alkyl, optionally substituted aryl and optionally substituted heteroaryl, and
R^{9′} is chosen from optionally substituted alkyl, optionally substituted aryl and optionally substituted heteroaryl;
or alternatively:
R⁶ taken together with R^{6′} is optionally substituted cycloalkyl or optionally substituted heterocycloalkyl having 5 to 7 ring atoms;
R⁶ and R⁷ taken together with the nitrogen to which R⁷ is bonded form an optionally substituted 5 to 12 membered heterocycle having up to 2 additional heteroatoms selected from O, N and S; or
R⁷ and R⁸, taken together with the nitrogen to which they are bonded form an optionally substituted 5 to 12 membered heterocycle having up to 2 additional heteroatoms selected from O, N and S; or
R⁶ taken together with R⁷, and R^{6′} taken together with R⁸ and the nitrogen to which R⁷ and R⁸ are bonded form an optionally substituted 10 to 13-membered fused heterocycle having up to 2 additional heteroatoms selected from O, N and S.

3. At least one chemical entity of claim 1 wherein
T is optionally substituted lower alkylene, and further having one or more of the following:
X, Y or Z is —N═, or W and Z are —N═;
R¹, R², R³ and R⁴ are independently hydrogen, halo, lower alkyl, substituted lower alkyl, lower alkoxy, cyano or absent;
R⁵ is other than optionally substituted phenyl;
R⁶ is lower alkyl;
R^{6′} is hydrogen;
R⁷ is substituted alkyl; and/or
R⁸ is —C(O)—R⁹, in which R⁹ is optionally substituted alkyl, optionally substituted aryl, optionally substituted aralkyl, aryloxyalkyl, optionally substituted heteroaryl, optionally substituted heteroaralkyl or optionally substituted heteroaryloxyalkyl;
R⁸ is —C(O)—OR^{9′}, in which R^{9′} is: optionally substituted aryl or optionally substituted heteroaryl;
R⁶ and R⁷ taken together with the nitrogen to which R⁷ is bonded form an optionally substituted 5 to 7 membered heterocycle having up to 2 additional heteroatoms selected from O, N and S; and
R⁷ and R⁸ taken together with the nitrogen to which they are bonded form an optionally substituted 5 to 7 membered heterocycle having up to 2 additional heteroatoms selected from O, N and S.

4. At least one chemical entity according to claim 1 wherein
R⁵ is other than optionally substituted phenyl when R⁶ is methyl, and optionally having one or more of the following:
R⁸ is —S(O)₂—R^{9′}, in which R^{9′} is: C₁-C₁₃ alkyl; heteroaryl; naphthyl; or phenyl optionally substituted with halo, lower alkyl, lower alkoxy, nitro, methylenedioxy, phenyl or trifluoromethyl; or
R⁸ is —CH₂—R⁹, in which R⁹ is: C₁-C₁₃ alkyl; substituted lower alkyl; benzyl; heterocyclyl; naphthyl; or phenyl optionally substituted with halo, lower alkyl, lower alkoxy, nitro, methylenedioxy, phenyl or trifluoromethyl; or
R⁷ taken together with R⁸ is 2-(optionally substituted)-4,4-(optionally di-substituted)-4,5-dihydro-imidazol-1-yl, 2-(optionally substituted phenyl)-imidazol-1-yl, or 4-(optionally substituted alkyl)-2-(optionally substituted aryl)-imidazol-1-yl;
optionally having one or more of the following:
X, Y or Z is —N═;
R¹, R², R³ and R⁴ are independently hydrogen, chloro, fluoro, lower alkyl substituted lower alkyl, methoxy, cyano or absent;
R⁵ is benzyl or substituted benzyl;
R⁶ is ethyl, i-propyl, c-propyl or t-butyl; and/or
R⁷ is a primary-amino-substituted lower alkyl, secondary-amino-substituted lower alkyl or tertiary-amino-substituted lower alkyl.

5. At least one chemical entity of claim 4 wherein
R⁹ is heterocyclyl; naphthyl; or phenyl substituted with halo, lower alkyl, lower alkoxy, nitro, methylenedioxy, phenyl or trifluoromethyl; or
R⁷ taken together with R⁸ is 2-(4-methylphenyl)-4,5-dihydro-imidazol-1-yl, 2-(3-fluoro-,4-methylphenyl)-4,5-dihydro-imidazol-1-yl, 2-(4-methylphenyl)-4,4-dimethyl-4,5-dihydro-imidazol-1-yl, 2-(3-fluoro-,4-methylphenyl)-4,4-dimethyl-4,5-dihydro-imidazol-1-yl, 2-phenyl-imidazol-1-yl, 2-p-toluoyl-imidazol-1-yl, 2-(4-fluorophenyl)-imidazol-1-yl, 2-(4-chlorophenyl)-imidazol-1-yl, 2-(3-fluoro-4-methylphenyl)-imidazol-1-yl, 4-(2-amino-ethyl)-2-phenyl-imidazol-1-yl, 4-(2-amino-ethyl)-2-p-tolyl-imidazol-1-yl, 4-(2-amino-ethyl)-2-(4-fluoro-phenyl)-imidazol-1-yl, 4-(2-amino-ethyl)-2-(4-chloro-phenyl)-imidazol-1-yl, 4-(2-amino-ethyl)-2-(3-fluoro-4-methyl-phenyl)-imidazol-1-yl, 4-(aminomethyl)-2-phenyl-imidazol-1-yl, 4-(aminomethyl)-2-p-tolyl-imidazol-1-yl, 4-(aminomethyl)-2-(4-fluoro-phenyl)-imidazol-1-yl, 4-(aminomethyl)-2-(4-chloro-phenyl)-imidazol-1-yl, or 4-(aminomethyl)-2-(3-fluoro-4-methyl-phenyl)-imidazol-1-yl.

6. At least one chemical entity of claim 1 wherein T is optionally substituted lower alkylene.

7. At least one chemical entity of claim 1 wherein T is C₁ to C₄ alkylene or C₁ to C₄ alkylene substituted with halo or oxo.

8. At least one chemical entity of claim 1 wherein T is C₁ to C₄ alkylene.

9. At least one chemical entity of claim 1 wherein T is absent.

10. At least one chemical entity of claim 1 wherein U is absent, C₁ to C₄ alkylene or C₁ to C₄ alkylene substituted with halo or oxo.

11. At least one chemical entity of claim 1 wherein U is absent.

12. At least one chemical entity of claim 1 wherein X, Y or Z is —N═, or W and Z are —N═.

13. At least one chemical entity of claim 1 wherein R¹, R², R³ and R⁴ are each independently chosen from hydrogen, halo, lower alkyl, substituted lower alkyl, lower alkoxy and cyano, or is absent.

14. At least one chemical entity of claim 1 wherein R¹, R², R³ and R⁴ are each independently hydrogen, chloro, fluoro, methyl, methoxy, cyano or substituted lower alkyl or is absent.

15. At least one chemical entity of claim 1 wherein R¹, R², R³ and R⁴ are each independently hydrogen, chloro, fluoro, methyl, methoxy or cyano or is absent.

16. At least one chemical entity of claim 1 wherein three or four of R¹, R², R³ and R⁴ are hydrogen.

17. At least one chemical entity of claim 1 wherein R¹, R², R³ and R⁴ are each hydrogen or is absent.

18. At least one chemical entity of claim 1 wherein three of R¹, R², R³ and R⁴ are hydrogen and the fourth is halo, methoxy, methyl, cyano or is absent.

19. At least one chemical entity of claim 18 wherein R¹, R², and R⁴ are hydrogen and R³ is chloro.

20. At least one chemical entity of claim 1 wherein R⁵ is optionally substituted aralkyl.

21. At least one chemical entity of claim 1 wherein R⁵ is benzyl or substituted benzyl.

22. At least one chemical entity of claim 1 wherein R⁵ is benzyl.

23. At least one chemical entity of claim 1 wherein R⁶ is optionally substituted lower alkyl and R^{6′} is hydrogen or optionally substituted lower alkyl.

24. At least one chemical entity of claim 1 wherein R^{6′} is hydrogen.

25. At least one chemical entity of claim 1 wherein R⁶ is C₃ to C₅ lower alkyl.

26. At least one chemical entity of claim 1 wherein R⁶ is i-propyl, c-propyl or t-butyl.

27. At least one chemical entity of claim 1 wherein R⁶ is i-propyl.

28. At least one chemical entity of claim 1 wherein R⁷ is substituted alkyl.

29. At least one chemical entity of claim 1 wherein R⁷ is alkyl substituted with a primary-, secondary- or tertiary-amine.

30. At least one chemical entity of claim 1 wherein R⁸ is —C(O)—R⁹ wherein R⁹ is: optionally substituted aryl, optionally substituted aralkyl, aryloxyalkyl, optionally substituted heteroaryl, optionally substituted heteroaralkyl, or optionally substituted heteroaryloxyalkyl.

31. At least one chemical entity of claim 1 wherein R⁹ is optionally substituted alkyl, optionally substituted aryl or optionally substituted aralkyl.

32. At least one chemical entity of claim 1 wherein R⁹ is lower alkoxyalkyl, lower alkyl-substituted phenyl, lower alkoxy-substituted phenyl, halo-substituted phenyl, optionally substituted benzyl, phenoxy lower alkyl, optionally substituted heteroaryl, optionally substituted heteroaralkyl or optionally substituted heteroaryloxyalkyl

33. At least one chemical entity of claim 1 wherein R⁹ is lower alkoxyalkyl or substituted phenyl.

34. At least one chemical entity of claim 1 wherein R⁹ is methoxy-methyl or p-tolyl.

35. At least one chemical entity of claim 1 wherein R⁸ is —C(O)—OR^{9′} and R^{9′} is optionally substituted aryl or optionally substituted heteroaryl.

36. At least one chemical entity of claim 1 wherein R⁶ and R⁷ taken together with U and the nitrogen to which R⁷ is bonded form an optionally substituted 5- to 12-membered heterocycle, optionally having up to 2 additional heteroatoms selected from O, N and S.

37. At least one chemical entity of claim 36 wherein T is not absent.

38. At least one chemical entity of claim 36 wherein T is absent and such 5- to 12-membered heterocycle is not optionally substituted: 5-oxo-pyrrolidin-2-yl, 6-oxo-piperid in-2-yl, 2-oxo-hexahydro-pyrimidin-4-yl, pyrrol id in-2-yl, pyrrolidin-3-yl, piperidin-2-yl, piperidin-3-yl or piperidin-4-yl.

39. At least one chemical entity of claim 1 wherein R⁷ and R⁸ taken together with the nitrogen to which they are bonded form an optionally substituted 5- to 12-membered heterocycle, optionally having up to 2 additional heteroatoms selected from O, N and S.

40. At least one chemical entity of claim 1 that is
N-(3-amino-propyl)-N-[1-(3-benzyl-4-oxo-3,4-dihydro-pyrido[2,3-d]pyrimidin-2-ylethyl-2-methyl-propyl]4-methyl-benzamide,
N-(3-amino-propyl)-N-[1-(3-benzyl-4-oxo-3,4-dihydro-pyrido[4,3-d]pyrimidin-2-ylethyl)-2-methyl-propyl]-4-methyl-benzamide,
N-(3-amino-propyl)-N-[1-(3-benzyl-4-oxo-3,4-dihydro-pyrido[3,4-d]pyrimidin-2-ylmethyl)-2-methyl-propyl]-4-methyl-benzamide,
N-(3-amino-propyl)-N-[1-(3-benzyl-4-oxo-3,4-dihydro-pyrido[2,3-d]pyrimidin-2-ylmethyl)-2-methyl-propyl]-4-methyl-benzamide,
2-{2-[6-(2-amino-ethyl)-3-methyl-1-(4-methyl-benzoyl)-piperidin-2-yl]-ethyl}-3-benzyl-5,6-dihydro-3H-pyrido[3,4-d]pyrimidin-4-one,
N-(3-amino-propyl)-N-{2-[(3-benzyl-4-oxo-3,4,5,7-tetrahydro-furo[3,4-d]pyrimidin-2-ylmethyl)-methyl-amino]-propyl}-4-methyl-benzenesulfonamide,
N-(3-amino-propyl)-N-[1-(3-benzyl-4-oxo-3,4-dihydro-pyrido[3,2-d]pyrimidin-2-ylpropyl)-2-methyl-propyl]-4-methyl-benzamide, or
N-(3-amino-propyl)-N-[1-(3-benzyl-4-oxo-3,4-dihydro-pteridin-2-ylmethyl)-2-methyl-propyl]-4-methyl-benzamide.

41. A pharmaceutical composition comprising at least one chemical entity of claim 1 and at least one pharmaceutically acceptable excipient.

42. A method of treating a cellular proliferative disease comprising administering at least one chemical entity of claim 1 in an effective amount to treat the cellular proliferative disease.

43. The method of claim 42 wherein the cellular proliferative disease is cancer.

44. (canceled)

45. (canceled)
